SEQUENCE LISTING (46 sequences)

SEQ ID NO: 1 - DNA, 34 bp, synthetic construct
  source 1..34: mol_type = other DNA
ataacttcgtatannntannntatacgaagttat

SEQ ID NO: 2 - DNA, 34 bp, synthetic construct
  source 1..34: mol_type = other DNA
ataacttcgtatagcatacattatacgaagttat

SEQ ID NO: 3 - DNA, 34 bp, synthetic construct
  source 1..34: mol_type = other DNA
cctagttcctatagcctacattataggatggagg

SEQ ID NO: 4 - DNA, 34 bp, synthetic construct
  source 1..34: mol_type = other DNA
agtagaacgtataggctaatttatacgatgtagg

SEQ ID NO: 5 - DNA, 34 bp, synthetic construct
  source 1..34: mol_type = other DNA
ataacttcgtatatggtattatatacgaagttat

SEQ ID NO: 6 - DNA, 34 bp, synthetic construct
  source 1..34: mol_type = other DNA
ataacttcgtatattctatcttatacgaagttat

SEQ ID NO: 7 - DNA, 34 bp, synthetic construct
  source 1..34: mol_type = other DNA
taccgggcgtatagcatacattatacgaagttat

SEQ ID NO: 8 - DNA, 34 bp, synthetic construct
  source 1..34: mol_type = other DNA
ataacttcgtatagcatacattatacgcccggta

SEQ ID NO: 9 - DNA, 34 bp, synthetic construct
  source 1..34: mol_type = other DNA
aatgcatgctatagcatacattatacgaagttat

SEQ ID NO: 10 - DNA, 34 bp, synthetic construct
  source 1..34: mol_type = other DNA
ataacttcgtatagcatacattataggtaccgag

SEQ ID NO: 11 - DNA, 34 bp, synthetic construct
  source 1..34: mol_type = other DNA
taccgggcgtatagcctacattatacgaagttat

SEQ ID NO: 12 - DNA, 34 bp, synthetic construct
  source 1..34: mol_type = other DNA
ataacttcgtatagcctacattatacgcccggta

SEQ ID NO: 13 - DNA, 34 bp, synthetic construct
  source 1..34: mol_type = other DNA
aatacatcgtataggctaatttatacgaagttat

SEQ ID NO: 14 - DNA, 34 bp, synthetic construct
  source 1..34: mol_type = other DNA
ataacttcgtataggctaatttataggtaccgag

SEQ ID NO: 15 - DNA, 68 bp, synthetic construct
  source 1..68: mol_type = other DNA
ataacttcgtatagcatacattatacgaagttattaccgggcgtatagcctacattatacgaagttat

SEQ ID NO: 16 - DNA, 68 bp, synthetic construct
  source 1..68: mol_type = other DNA
ataacttcgtatagcatacattatacgaagttatataacttcgtatagcctacattatacgcccggta

SEQ ID NO: 17 - DNA, 68 bp, synthetic construct
  source 1..68: mol_type = other DNA
ataacttcgtatagcatacattatacgaagttataatacatcgtataggctaatttatacgaagttat

SEQ ID NO: 18 - DNA, 68 bp, synthetic construct
  source 1..68: mol_type = other DNA
ataacttcgtatagcatacattatacgaagttatataacttcgtataggctaatttataggtaccgag

SEQ ID NO: 19 - DNA, 13 bp, synthetic construct
  source 1..13: mol_type = other DNA
ataacttcgtata

SEQ ID NO: 20 - skipped

SEQ ID NO: 21 - DNA, 13 bp, synthetic construct
  source 1..13: mol_type = other DNA
tatacgaagttat

SEQ ID NO: 22 - DNA, 13 bp, synthetic construct
  source 1..13: mol_type = other DNA
cctagttcctata

SEQ ID NO: 23 - skipped

SEQ ID NO: 24 - DNA, 13 bp, synthetic construct
  source 1..13: mol_type = other DNA
tataggatggagg

SEQ ID NO: 25 - DNA, 13 bp, synthetic construct
  source 1..13: mol_type = other DNA
agtagaacgtata

SEQ ID NO: 26 - skipped

SEQ ID NO: 27 - DNA, 13 bp, synthetic construct
  source 1..13: mol_type = other DNA
tatacgatgtagg

SEQ ID NO: 28-29 - skipped (2 entries)

SEQ ID NO: 30 - DNA, 13 bp, synthetic construct
  source 1..13: mol_type = other DNA
taccgggcgtata

SEQ ID NO: 31 - DNA, 13 bp, synthetic construct
  source 1..13: mol_type = other DNA
tatacgcccggta

SEQ ID NO: 32 - DNA, 13 bp, synthetic construct
  source 1..13: mol_type = other DNA
aatgcatgctata

SEQ ID NO: 33 - DNA, 13 bp, synthetic construct
  source 1..13: mol_type = other DNA
tataggtaccgag

SEQ ID NO: 34 - DNA, 10 bp, synthetic construct
  source 1..10: mol_type = other DNA
tgctggcctt

SEQ ID NO: 35 - skipped

SEQ ID NO: 36 - DNA, 78 bp, synthetic construct
  source 1..78: mol_type = other DNA
ataacttcgtatagcatacattatacgaagttattgctggccttataacttcgtatagcctacattatacgcccggta

SEQ ID NO: 37 - DNA, 13 bp, synthetic construct
  source 1..13: mol_type = other DNA
aatacatcgtata

SEQ ID NO: 38 - DNA, 78 bp, synthetic construct; identical to SEQ ID NO: 36
  source 1..78: mol_type = other DNA

SEQ ID NO: 39 - DNA, 34 bp, synthetic construct
  source 1..34: mol_type = other DNA
taccgggcgtatagcctacattatacgcccggta

SEQ ID NO: 40 - DNA, 34 bp, synthetic construct
  source 1..34: mol_type = other DNA
aatacatcgtataggctaatttataggtaccgag

SEQ ID NO: 41 - DNA, 78 bp, synthetic construct
  source 1..78: mol_type = other DNA
ataacttcgtatagcatacattatacgaagttattgctggccttaatacatcgtataggctaatttatacgaagttat

SEQ ID NO: 42 - DNA, 78 bp, synthetic construct
  source 1..78: mol_type = other DNA
ataacttcgtatagcatacattatacgaagttattgctggccttataacttcgtataggctaatttataggtaccgag

SEQ ID NO: 43 - DNA, 34 bp, synthetic construct
  source 1..34: mol_type = other DNA
taccgggcgtatatggtattatatacgaagttat

SEQ ID NO: 44 - DNA, 34 bp, synthetic construct
  source 1..34: mol_type = other DNA
ataacttcgtatatggtattatatacgcccggta

SEQ ID NO: 45 - DNA, 34 bp, synthetic construct
  source 1..34: mol_type = other DNA
aatacatcgtatattctatcttatacgaagttat

SEQ ID NO: 46 - DNA, 34 bp, synthetic construct
  source 1..34: mol_type = other DNA
ataacttcgtatattctatcttataggtaccgag

(19) United States
(12) Patent Application Publication
(10) Pub. No.: US 2026/0265768 A1
(43) Pub. Date: Sep. 10, 2026

(54) COMPOSITIONS AND METHODS FOR TARGETABLE GENETIC RECOMBINATION IN PLANTS

(71) Applicant: University of Georgia Research Foundation, Inc., Athens, GA (US)

(72) Inventors: Katie Toomey, Athens, GA (US); Peter LaFayette, Athens, GA (US); Wayne Parrott, Athens, GA (US)

(21) Appl. No.: 19/557,016

(22) Filed: Mar. 4, 2026

Related U.S. Application Data
(60) Provisional application No. 63/766,625, filed on Mar. 4, 2025.

(51) Int. Cl. C12N 15/82; C12N 5/04; C12N 9/12

(52) U.S. Cl. CPC C12N 15/8216; C12N 5/04; C12N 9/1241; C12N 15/8213; C12N 2800/30; C12N 2830/34

(57) ABSTRACT

Predictable and reliable gene stacking will simplify the breeding process, saving time and resources. The Cre-Lox recombination system offers the ability to easily add, remove, or alter traits for more precise control over plant characteristics. The system uses specific bespoke loxP sites to ensure unidirectional gene insertion, creating a “Loxbox” to prevent further insertions at the same site. This method allows for iterative, targeted gene stacking approach to user-defined genetic engineering by recombination in a plant cell, plant tissue, plant material, plant part, and/or plant with predictable directionality. By creating a toolkit for targeted sequential gene insertion, the trait development pipeline can be streamlined, making it easier and faster to develop new plant varieties with desirable traits.

### CROSS-REFERENCE TO RELATED APPLICATIONS

This application claims the benefit of and priority to U.S. Application No. 63/766,625 filed Mar. 4, 2025, the content of which is incorporated by reference herein in its entirety.

### STATEMENT REGARDING FEDERALLY SPONSORED RESEARCH

This invention was made with government support under grant DE-SC0023338 awarded by the United States Department of Energy. The government has certain rights in the invention.

### REFERENCE TO THE SEQUENCE LISTING

The Sequence Listing submitted as an XML file named “UGA_2025-124-02_ST26.xml”, created on Mar. 4, 2026, and having a size of 37,922 bytes is hereby incorporated by reference pursuant to 37 C.F.R. § 1.834 (c) (1).

### FIELD OF THE INVENTION

The disclosed invention is generally in the field of genetically engineered plants and specifically in the area of initiating and monitoring genetic recombination events.

### BACKGROUND OF THE INVENTION

Repetitive targeted gene insertions can consolidate multiple constructs into a single locus, known as gene stacking, making breeding segregation more predictable compared to techniques that rely upon dispersed genomic insertions. This gene-stacking method has been successfully implemented in maize (Ainley et al., 2013; Cody et al., 2020; Kumar et al., 2015) and soybean (Bonawitz et al., 2019) with technologies like ZFN and NHEJ, though these have limitations. ZFN is labor-intensive, and NHEJ is unreliable for plant insertions (Dong & Ronald, 2021). Consequently, some researchers stack genes in bacteria before plant transformation (Collier et al., 2018; Hathwaik et al., 2021; McCue et al., 2019; Zhu et al., 2017). This one-step transformation accelerates plant transformation and is a visible approach if the final construct is well-tested and unlikely to change. However, this method lacks flexibility as any alteration to the final construct requires returning to cloning and re-targeting the multi-construct assembly. New approaches to enhance the reliability and predictability of gene expression within plants are needed to ensure the security of agriculture and food production.

Given the limitations associated with conventional plant-genetic manipulation methods, there is a need for enhancing the genetic manipulation of plants that can facilitate predictable breeding and segregation.

Therefore, it is an object of the invention to provide compositions and methods that enable iterative, targeted gene stacking in plant cells, plant tissues, plant materials, plant parts, and plants with predictable directionality.

It is a further object of the invention to provide compositions and methods that enable a flexible approach for unidirectional insertion(s) of gene(s) within plant cells, plant tissues, plant materials, plant parts, and/or plants, that prevents further insertions at the same site.

It is a further object of the invention to provide compositions and methods that readily enable user-defined addition, removal, or alteration of traits for precise control over characteristics of plant cells, plant tissues, plant materials, plant parts, and plants.

### BRIEF SUMMARY OF THE INVENTION

A coordinated, multi-recombinase site framework, optimized for iterative transformations in planta has been developed. Compositions of Cre-Lox recombination elements that include specific bespoke loxP sites have been developed for unidirectional gene insertion within a plant cell, plant tissue, plant material, plant part, or plant. The compositions provide a “Loxbox” element to ensure gene insertion within a target plant cell's genome at a user-defined location. The structure of the Loxbox composition is configured to preclude further undesired genetic modifications, such as further insertions at the same site. Cre-Lox recombination systems for transformation and breeding are also provided, which involve designed sets of loxP mutants that limit (but do not preclude) Cre-mediated recombination, and which themselves recombine to form non-functional loxP double-mutant (“loxbox”) sequences, which are not functionally active to mediate subsequent Cre-based recombination. Methods for gene stacking in plants are also described. Typically, the methods implement step-wise sequential recombination events to give rise to the double-mutant “loxbox” loxP sequences, which permit the introduction of subsequent recombinant genes within the same genomic region without the risk of undesired, non-specific recombination.

A modified plant genome including a mutant loxP landing pad nucleotide sequence is provided, where the mutant loxP landing pad includes
(i) a wild-type loxP nucleotide sequence, optionally where the wild-type loxP includes SEQ ID NO: 1 or SEQ ID NO:2; and
(ii) a first mutant loxP nucleotide sequence, optionally where the first mutant loxP nucleotide sequence includes a nucleic acid sequence set forth in any one of SEQ ID NOs: 3-14. In some forms, the wild-type loxP nucleotide sequence is contiguous with the first mutant loxP nucleotide sequence. In some forms, the wild-type loxP nucleotide sequence includes any one of SEQ ID NOs: 15-18.

A recombination mediated exchange cassette (RMEC) for genetic modification of the plant genome is also provided, including
(i) a wild-type loxP nucleotide sequence, optionally where the wild-type loxP includes SEQ ID NO:1 or SEQ ID NO:2;
(ii) a second mutant loxP nucleotide sequence;
(iii) a donor nucleotide sequence; and
(iv) a third mutant loxP nucleotide sequence;
where the second and third mutant loxP nucleotide sequence(s) are orientated either side of the donor nucleotide sequence; and where the wild-type loxP nucleotide sequence and the second mutant loxP nucleotide sequence are orientated upstream of the donor nucleotide sequence; and where the third mutant loxP sequence is configured to direct Cre recombinase-mediated recombination with the first mutant loxP sequence within the landing pad of the modified plant genome. In some forms each of the first, second and third mutant loxP sequences includes a left arm region, a right arm region and a spacer sequence, where each of the first, second and third mutant loxP sequences include a variation of a wildtype loxP sequence in a left arm region or in a right arm region, optionally where each variation includes a substitution of one or more nucleotides in the left arm region or the right arm region. In some forms the donor nucleotide sequence includes (i) one or more gene, optionally wherein the gene is exogenous to the target plant genome; and/or (ii) a reporter gene. In some forms the first mutant loxP nucleotide sequence includes any one of SEQ ID NOs: 3, 5, 7, 9, 11, or 13; and the third mutant loxP nucleotide sequence includes any one of SEQ ID NOs: 4, 6, 8, 10, 12, or 14; and the second mutant loxP nucleotide sequence is not the same as the first or third mutant loxP nucleotide sequence. In some forms the second mutant loxP nucleotide sequence includes any one of SEQ ID NOs: 3-14. In some forms the first mutant loxP nucleotide sequence includes SEQ ID NO:11; and the third mutant loxP nucleotide sequence includes SEQ ID NOs: 12. In some forms the second mutant loxP nucleotide sequence includes any one of SEQ ID NO: 13 or 14.

In some forms the first mutant loxP nucleotide sequence includes SEQ ID NO:12; and the third mutant loxP nucleotide sequence includes SEQ ID NO:11. In some forms second mutant loxP nucleotide sequence includes SEQ ID NO:13. In some forms the RMEC further includes a promoter and/or a marker gene. In some forms the RMEC further includes an AmCyan gene, or a YFP gene, or both. In some forms the RMEC further includes an AmCyan gene and/or the YFP gene that is not operably linked to a promoter, and is configured within the RMEC to become operably linked to a promoter upon recombination into a recipient plant genome. A plant cell, plant tissue, plant material, plant part, or plant including the described RMEC(s) is also provided.

Methods for iterative stacking of donor genes within a target plant genome are also described. Typically, the methods include the steps of (a) contacting a target plant genome including a first mutant loxP landing pad with a first recombination mediated exchange cassette (RMEC), including: (i) a wild-type loxP nucleotide sequence, optionally where the wild-type loxP includes SEQ ID NO: 1 or SEQ ID NO:2; (ii) a second mutant loxP nucleotide sequence; (iii) a donor nucleotide sequence; and (iv) a third mutant loxP nucleotide sequence; where the second and third mutant loxP nucleotide sequence(s) are orientated either side of the donor nucleotide sequence within the RMEC; and where the wild-type loxP nucleotide sequence and the second mutant loxP nucleotide sequence are orientated upstream of the donor nucleotide sequence within the RMEC; and where the third mutant loxP sequence is configured to direct Cre recombinase-mediated recombination with the first mutant loxP sequence within the first landing pad, where the contacting occurs under conditions that enable Cre-recombinase mediated exchange of the RMEC into the first landing pad to provide a second mutant loxP landing pad including the second mutant loxP sequence; and (b) optionally confirming recombination of the donor nucleotide sequence into the target genome. In some forms, the methods also include (c) contacting the target plant genome including the second mutant loxP landing pad with a second recombination mediated exchange cassette (RMEC), including (i) a wild-type loxP nucleotide sequence, optionally where the wild-type loxP includes SEQ ID NO: 1 or SEQ ID NO:2; and (ii) a fourth mutant loxP nucleotide sequence; and (iii) a donor nucleotide sequence; and (iv) a fifth mutant loxP nucleotide sequence; where the fourth and fifth mutant loxP nucleotide sequence(s) are orientated either side of the donor nucleotide sequence within the RMEC; and where the wild-type loxP nucleotide sequence and the fifth mutant loxP nucleotide sequence are orientated upstream of the donor nucleotide sequence within the RMEC; and where the fourth mutant loxP sequence is configured to direct Cre recombinase-mediated recombination with the second mutant loxP sequence within the second landing pad; where the contacting occurs under conditions that enable Cre-recombinase mediated exchange of the RMEC into the second landing pad to provide a third mutant loxP landing pad including the fifth mutant loxP sequence; and (e) optionally confirming recombination of the donor nucleotide sequence into the target genome. In some forms, the methods also include (f) repeating one or more of steps (a)-(d) once or more than once to iteratively recombine donor gene(s) into the target plant genome. In some forms, the methods also include, prior to step (a), inserting within a target plant genome a first mutant loxP landing pad nucleotide sequence, where the mutant loxP landing pad includes (i) a wild-type loxP nucleotide sequence, optionally where the wild-type loxP includes SEQ ID NO: 1 or SEQ ID NO: 2; and (ii) a first mutant loxP nucleotide sequence; optionally where the first mutant loxP nucleotide sequence includes a nucleic acid sequence set forth in any one of SEQ ID NOs: 3-14. A modified plant cell, plant tissue, plant material, plant part, or plant prepared according to the described methods is also provided. A modified plant genome including a nucleic acid sequence including one or more heterologous genes derived from Cre-mediated recombination and one or more LoxBox sequences is also provided. In some forms, the one or more LoxBox sequence includes any one of SEQ ID NOs: 39 or 40.

Methods for developing genetically modified plant cells, plant tissues, plant materials, plant parts, and plants including the described genetic modifications are also provided. The methods provide for iterative, targeted gene stacking with predictable directionality in plant cells, plant tissues, plant materials, plant parts, and plants. In some forms, the methods include one or more steps for creating a “toolkit” for targeted sequential gene insertion. In some forms, the methods include one or more steps to provide a “trait development pipeline”, including providing a plurality of different genetically modified plant cells, plant tissues, plant materials, plant parts, and plants that include one or more user-defined genetic modifications that introduce the same or different trait(s) within a plant cell, plant tissue, plant material, plant part, and/or plant of the plurality. For example, in some forms, the methods enhance the speed and efficacy of the development of a new plant variety having one or more desirable trait(s).

Genetically modified plant cells, plant tissues, plant materials, plant parts, and plants prepared according to the described methods area also provided. In some forms, the genetically modified plant cells, plant tissues, plant materials, plant parts, and/or plants include one or more traits, such as a desired trait.

Components or parts of the genetically modified plants, such as seeds, fruits or flowers are also provided. Offspring plants derived from breeding of the genetically modified plants are also provided. In some forms, the genetically modified plant has increased yield, or has increased resilience to a pest or environmental condition, In some forms, the plant is a crop plant associated with food production.

### BRIEF DESCRIPTION OF THE DRAWINGS

FIGS. 1A-1C are schematics of wildtype and mutant loxP sequences, showing left and right arm mutants. FIG. 1A is a legend showing the different components required for loxP-based recombination events. FIG. 1B is a schematic showing compatibility of mutant loxP sequence pairs. FIG. 1C is a schematic showing specificity of mutant loxP sequence pairs.

FIGS. 2A-2C are schematics of recombinase-mediated cassette exchange, including initial transformation; and first donor introduction (FIG. 2A); recombination and Second donor introduction (FIG. 2B); and Cycle continuation (FIG. 2C).

FIG. 3 is a flow-chart depicting analytical methods.

FIGS. 4A-4B show schematics of recombinase-mediated cassette exchange (RMCE), with two wt loxPs that can insert forward or reverse (FIG. 4A); RMCE with two pairs of recombinase sites with differing spacers and arms so only one direction is feasible. Note that the fully unshaded triangle represents a “Loxbox” (FIG. 4B).

FIGS. 5A-5C show schematics of alternating recombinase pairs for RMCE. FIG. 5A, is a legend depicting a wild-type (shaded) loxP, with wild-type spacer arms; a differentially part-shaded first loxP pair, having specific spacers and mutated arms, and a differentially part-shaded second loxP pair, respectively. FIG. 5B shows a first loxP landing pad 1 and a corresponding donor cassette configured with a corresponding first loxP sequence, which undergoes RMCE in contact with Cre to insert the donor cassette. FIG. 5C shows a first loxP landing pad and a corresponding donor cassette configured with a corresponding first loxP sequence as well as a second loxP configured to include a second loxP sequence, which undergoes RMCE in contact with Cre to insert the donor cassette and also to create a new landing pad 2 within the recombined genome at the user-defined position.

FIGS. 6A-6B show schematics of loxP excision for a transient (FIG. 6A) assay, and stable (FIG. 6B) assay, respectively. FIG. 6A shows loxP excision process for transient assays, with a marker (YFP) expression from within a wtloxP-wtloxP region following initial transformation, and subsequent Cre excision of the wtloxP-wtloxP region, leaving a single recombined wtloxP that does not express YFP. FIG. 6B shows loxP excision process for stable transformations, with induction of Cre with heat that would excise the loxP-loxP region leaving a recombined loxP in its place. The 2×CsVMV promoter that was driving YFP will instead drive AmCyan.

FIGS. 7A-7B show schematics of loxP compatibility and specificity profiles. FIG. 7A is a schematic showing all possible compatibility tests to conduct based on wild-type (LCL) and the 4 new pairs of recombinase sites, respectively. (FIG. 7B) is a schematic showing all possible specificity tests to conduct based on wild-type (LCL) and the 4 new recombinase sites.

FIGS. 8A-8C show schematics of iterative RMCE procedure. FIG. 8A shows the initial transformation will contain YFP and nptII selection as well as the landing pad composed of loxP and KitR. The black line that the construct is on represents the genome and the first donor for RMCE begins with wt loxP and ends with KitL. It contains the visual marker Amcyan that lacks a promoter. There is room for a gene of interest (GOI) to be between the terminator of AmCyan and the KitL; FIG. 8B shows when KitR and KitL recombine, they will form KitLR which is a double mutant “loxbox” After the first RMCE, the 2×CsVMV which was driving YFP would now drive AmCyan. The second donor will contain a YFP that lacks a promoter. When the donor is inserted with the second gene of interest and YFP, the upstream 2×CsVMV will drive the YFP. Alternating between the AmCyan and YFP markers allows for visual aid in selecting the RMCE events. FIG. 8C shows that the resulting recombination leaves two “Loxboxes” in the genome and the same landing pad as the first insertion, thus allowing the cycle to begin again.

FIGS. 9A-9C are graphs depicting emission spectra for AmCyan (FIG. 9A), and for YFP (FIG. 9B). NIGHTSEA offers handheld and microscope-mounted wavelength sets that work for AmCyan (VI) and YFP (CY) without overlap (FIG. 9C). This allows for discrimination between the two.

FIGS. 10A-10B depict the design and data of a transient assay. FIG. 10A is a schematic depicting the test constructs. FIG. 10B is a graph depicting the relative excision efficacy of constructs being tested as a transient assay. The relative excision efficiency of the pairs as normalized by LCL. LCL was left in the graph to show how the other constructs compare.

FIGS. 11A-11C depict the design and data of a transient assay. FIG. 11A is a schematic depicting the test constructs. FIG. 11B is a graph depicting the relative excision efficacy of constructs being tested as a transient assay. FIG. 11C is a graph depicting the The standardized excision efficiency of the specificity constructs is as normalized by LCL. LCL was left in the graph to show how the other constructs compare.

FIGS. 12A-12C are schematics depicting RMCE constructs, including construct transformed into poplar 717 stably (FIG. 12A); the various RMCE donor #1 (D1) constructs available, containing AmCyan (DIA), AmCyan fused to HPH (D1AH), and containing HPH alone (D1H), respectively, in poplar and N. benthamiana and contain 3 constructs as genes of interest (GOI) listed on the right (FIG. 12B); and the RMCE donor #2 (D2) containing Yukon as well as two genes of interest listed on the right (FIG. 12C).

FIG. 13 is a graph depicting relative excision efficacy of constructs ested as a transient assay. The relative excision efficiency of the pairs as normalized by LCL. LCL was left in the graph to show how the other constructs compare.

FIGS. 14A-14B are schematics depicting Flp/FRT excision by flanking the donor cassette with FRT sites (FIG. 14A); the donor is excised from its chromosomal context upon FLP expression, which would then allow the donor to exist as an extrachromosomal circle, thus improving accessibility (FIG. 14B).

### DETAILED DESCRIPTION OF THE INVENTION

### I. Definitions

As used herein, the term “plant” is used in its broadest sense. It includes, but is not limited to, any species of woody, ornamental or decorative crop or cereal, and fruit or vegetable plant. It also refers to a plurality of plant cells that are largely differentiated into a structure that is present at any stage of a plant's development. Such structures include, but are not limited to, a fruit, shoot, stem, leaf, flower petal, etc.

As used herein, the term “plant tissue” includes differentiated and undifferentiated tissues of plants including those present in roots, shoots, leaves, pollen, seeds and tumors, as well as cells in culture (e.g., single cells, protoplasts, embryos, callus, etc.). Plant tissue may be in planta, in organ culture, tissue culture, or cell culture. The term “plant part” as used herein refers to a plant structure, a plant organ, or a plant tissue.

As used herein, the term “plant material” refers to leaves, stems, roots, flowers or flower parts, fruits, pollen, egg cells, zygotes, seeds, cuttings, cell or tissue cultures, or any other part or product of a plant.

As used herein, the term “plant part” or “part of a plant” can include, but is not limited to cuttings, cells, protoplasts, cell tissue cultures, callus (calli), cell clumps, embryos, stamens, pollen, anthers, pistils, ovules, flowers, seed, petals, leaves, stems, and roots.

As used herein, the term “plant organ” refers to a distinct and visibly structured and differentiated part of a plant such as a root, stem, leaf, flower bud, or embryo.

As used herein, the term “plant cell” refers to a structural and physiological unit of a plant, comprising a protoplast and a cell wall. The plant cell may be in form of an isolated single cell or a cultured cell, or as a part of higher organized unit such as, for example, a plant tissue, a plant organ, or a whole plant.

As used herein, the term “plant cell culture” refers to cultures of plant units such as, for example, protoplasts, cell culture cells, cells in plant tissues, pollen, pollen tubes, ovules, embryo sacs, zygotes and embryos at various stages of development.

As used herein, a “cultivar” refers to a cultivated variety.

As used herein, “germplasm” refers to one or more phenotypic characteristics, or one or more genes encoding said one or more phenotypic characteristics, capable of being transmitted between generations.

As used herein, the term “progenitor” refers to any of the species, varieties, cultivars, or germplasm, from which a plant is derived.

As used herein, the term “derivative species, germplasm or variety” refers to any plant species, germplasm or variety that is produced using a stated species, variety, cultivar, or germplasm, using standard procedures of sexual hybridization, recombinant DNA technology, tissue culture, mutagenesis, or a combination of any one or more said procedures.

As used herein, a “hybrid” is typically derived from one or more crosses between different varieties, germplasms, populations, breeds or cultivars within a single species, between different subspecies within a species, or between different species within a genus. Typically, hybrids between subspecies are referred to as “intra-specific hybrids” and hybrids between different species within a genus are referred to as “interspecific hybrids.”

As used herein, the term “transgenic plant” refers to a plant or tree that contains recombinant genetic material not normally found in plants or trees of this type and which has been introduced into the plant in question (or into progenitors of the plant) by human manipulation. Thus, a plant that is grown from a plant cell into which recombinant DNA is introduced by transformation is a transgenic plant, as are all offspring of that plant that contain the introduced transgene (whether produced sexually or asexually). It is understood that the term transgenic plant encompasses the entire plant or tree and parts of the plant or tree, for instance grains, seeds, flowers, leaves, roots, fruit, pollen, stems etc.

As used herein, the term “construct” refers to a recombinant genetic molecule having one or more isolated polynucleotide sequences. Genetic constructs used for transgene expression in a host organism include in the 5′-3′ direction, a promoter sequence; a sequence encoding a gene of interest; and a termination sequence. The construct may also include selectable marker gene(s) and other regulatory elements for expression.

As used herein, the term “gene” refers to a DNA sequence that encodes through its template or messenger RNA a sequence of amino acids characteristic of a specific peptide, polypeptide, or protein. The term “gene” also refers to a DNA sequence that encodes an RNA product. The term gene as used herein with reference to genomic DNA includes intervening, non-coding regions as well as regulatory regions and can include 5′ and 3′ ends.

As used herein, the term “orthologous genes” or “orthologs” refer to genes that have a similar nucleic acid sequence because they were separated by a speciation event.

As used herein, the term, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

As used herein, the term “isolated” is meant to describe a compound of interest (e.g., nucleic acids) that is in an environment different from that in which the compound naturally occurs, e.g., separated from its natural milieu such as by concentrating a peptide to a concentration at which it is not found in nature. “Isolated” is meant to include compounds that are within samples that are substantially enriched for the compound of interest and/or in which the compound of interest is partially or substantially purified. Isolated nucleic acids are at least 60% free, preferably 75% free, and most preferably 90% free from other associated components. An “isolated” nucleic acid molecule or polynucleotide is a nucleic acid molecule that is identified and separated from at least one contaminant nucleic acid molecule with which it is ordinarily associated in the natural source. The isolated nucleic acid can be, for example, free of association with all components with which it is naturally associated. An isolated nucleic acid molecule is other than in the form or setting in which it is found in nature.

As used herein, the term “locus” refers to a specific position along a chromosome or DNA sequence. Depending upon context, a locus could be a gene, a marker, a chromosomal band or a specific sequence of one or more nucleotides.

As used herein, the term “allele” refers to one of two or more alternative forms of a gene.

As used herein, the term “vector” refers to a replicon, such as a plasmid, phage, or cosmid, into which another DNA segment may be inserted so as to bring about the replication of the inserted segment. The vectors can be expression vectors.

As used herein, the term “expression vector” refers to a vector that includes one or more expression control sequences.

As used herein, the term “expression control sequence” refers to a DNA sequence that controls and regulates the transcription and/or translation of another DNA sequence. Control sequences that are suitable for prokaryotes, for example, include a promoter, optionally an operator sequence, a ribosome binding site, and the like. Eukaryotic cells are known to utilize promoters, polyadenylation signals, and enhancers.

As used herein, the term “promoter” refers to a regulatory nucleic acid sequence, typically located upstream (5′) of a gene or protein coding sequence that, in conjunction with various elements, is responsible for regulating the expression of the gene or protein coding sequence. The promoters suitable for use in the constructs of this disclosure are functional in plants and in host organisms used for expressing the disclosed polynucleotides. Many plant promoters are publicly known. These include constitutive promoters, inducible promoters, tissue- and cell-specific promoters and developmentally-regulated promoters. Exemplary promoters and fusion promoters are described, e.g., in U.S. Pat. No. 6,717,034, which is herein incorporated by reference in its entirety.

As used herein, a nucleic acid sequence or polynucleotide is “operably linked” when it is placed into a functional relationship with another nucleic acid sequence. For example, DNA for a presequence or secretory leader is operably linked to DNA for a polypeptide if it is expressed as a preprotein that participates in the secretion of the polypeptide; a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous and, in the case of a secretory leader, contiguous and in reading frame. Linking can be accomplished by ligation at convenient restriction sites. If such sites do not exist, synthetic oligonucleotide adaptors or linkers are used in accordance with conventional practice.

As used herein, the terms “transformed,” “transfected” “recombinant” and “engineered” refer to a host organism such as a bacterium or a plant into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Engineered cells, tissues, or plants are understood to encompass not only the end product of a transformation process, but also progeny, e.g., transgenic progeny, thereof. A “non-transformed,” “non-recombinant” or “unmodified” host refers to a wild-type organism, e.g., a bacterium or plant, which does not contain the heterologous nucleic acid molecule.

As used herein, the term “endogenous” with regard to a nucleic acid refers to nucleic acids normally present in the host.

As used herein, the term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. The term “heterologous” thus can also encompasses “exogenous” and “non-native” elements.

As used herein, “homologous” means derived from the same species. For example, a homologous trait is any characteristic of organisms that is derived from a common ancestor. Homologous sequences can be orthologous or paralogous. Homologous sequences are orthologous if they were separated by a speciation event: when a species diverges into two separate species, the divergent copies of a single gene in the resulting species are said to be orthologous. Orthologs, or orthologous genes, are genes in different species that are similar to each other because they originated from a common ancestor. Homologous sequences are paralogous if they were separated by a gene duplication event: if a gene in an organism is duplicated to occupy two different positions in the same genome, then the two copies are paralogous.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell.

Recitation of ranges of values herein are merely intended to serve as a shorthand method of referring individually to each separate value falling within the range, unless otherwise indicated herein, and each separate value is incorporated into the specification as if it were individually recited herein.

Use of the term “about” is intended to describe values either above or below the stated value in a range of approx.+/−10%; in other forms the values may range in value either above or below the stated value in a range of approx.+/−5%; in other forms the values may range in value either above or below the stated value in a range of approx.+/−2%; in other forms the values may range in value either above or below the stated value in a range of approx.+/−1%. The ranges are intended to be made clear by context, and no further limitation is implied.

The use of any and all examples, or exemplary language (e.g., “such as”) provided herein, is intended merely to better illuminate the description and does not pose a limitation on the scope of the description unless otherwise claimed.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed that while specific reference of each various individual and collective combinations and permutation of these compounds may not be explicitly disclosed, each is specifically contemplated and described herein. For example, if a ligand is disclosed and discussed and a number of modifications that can be made to a number of molecules including the ligand are discussed, each and every combination and permutation of ligand and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. Further, each of the materials, compositions, components, etc. contemplated and disclosed as above can also be specifically and independently included or excluded from any group, subgroup, list, set, etc. of such materials.

These concepts apply to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

All methods described herein can be performed in any suitable order unless otherwise indicated or otherwise clearly contradicted by context. The use of any and all examples, or exemplary language (e.g., “such as”) provided herein, is intended merely to better illuminate the embodiments and does not pose a limitation on the scope of the embodiments unless otherwise claimed. No language in the specification should be construed as indicating any non-claimed element as essential to the practice of the invention.

### II. Engineered Plant Cells, Plant Tissues, Plant Materials, Plant Parts, And Plants

It has been discovered that loxP specific recombination at specific sites within a plant cell's genome can be used for iterative targeted gene insertion in plant cells, plant tissues, plant materials, plant parts, and plants.

### A. loxP-Cre

“Target” genes of interest are more useful for breeding purposes when “stacked” in a predictable and unidirectional manner at a predetermined genetic location. loxP-specific recombination can be mediated with the Cre recombinase protein and its target site loxP to implement DNA modification targeted to a specific cell type, and/or triggered by a specific external stimulus. The Cre-lox system requires a single enzyme, Cre recombinase, that recombines a pair of short LoxP target sequences. This system can be implemented without inserting any extra supporting proteins or sequences. The Cre enzyme and the original Lox site called the LoxP sequence are derived from bacteriophage P1. Appropriate placement of Lox sequences allows genes to be activated, repressed, or exchanged for other genes. The activity of the Cre enzyme can be controlled so that it is expressed in a particular cell type or triggered by an external stimulus like a chemical signal or a heat shock.

loxP (locus of X-over P1) is a site on the bacteriophage P1 consisting of approximately 34 nucleotides. An exemplary loxP site includes an asymmetric 8 nucleotide “spacer” sequence, variable except for the middle two bases, in between two sets of symmetric, 13 nucleotide “arm” sequences.

ATAACTTCGTATANNNTANNNTATACGAAGTTAT (SEQ ID NO:1; “Wildtype” LoxP site; the left arm region is indicated in bold; and the spacer region is indicated in italics; and the right arm region is indicated in normal text; “N” is any nucleotide).

In some forms, the LoxP site is a mutant LoxP site. For example, in some forms, mutation(s) in the “spacer” region and one or both of arms of a loxP site can alter the recombination associated with the loxP site. “Variant”, or “mutant” loxP sites may include more or less nucleotides than WT loxP.

When the “wildtype” loxP site is employed, Cre activity is bidirectional with regard to recombination events.

The sequence of the spacer region determines directed recombination. When the spacer region includes a mutation, there may be only limited or no recombination with a wildtype loxP site, but there may be recombination with a corresponding mutant spacer sequence. Some spacer mutations can lead to limited recombination, others can lead to greater recombination with other spacers.

The sequence of the “arm” regions determines Cre recognition and subsequent enzyme activity. Cre can tolerate mutations in one arm, but not both.

1. loxP-Cre Mutants

Engineered loxP-Cre mutant systems for iterative, targeted gene insertion in plant cells, plant tissues, plant materials, plant parts, and plants including compatible loxP pairs are provided. In some forms, the loxp pairs are designed for highly specific recombination of plant cell, plant tissue, plant material, plant part, and/or plant genes in the presence of a functional Cre enzyme. The mutant loxPs are unable to recombine with non-matching loxP sites, even in the presence of functional Cre.

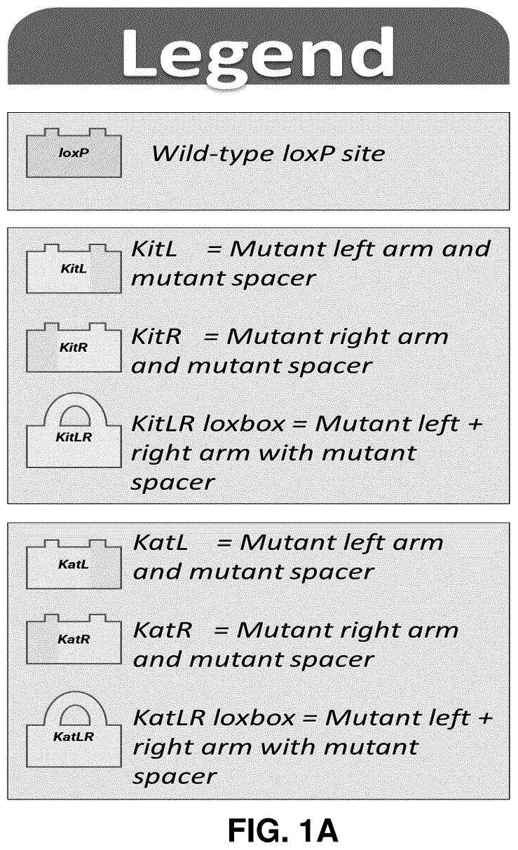

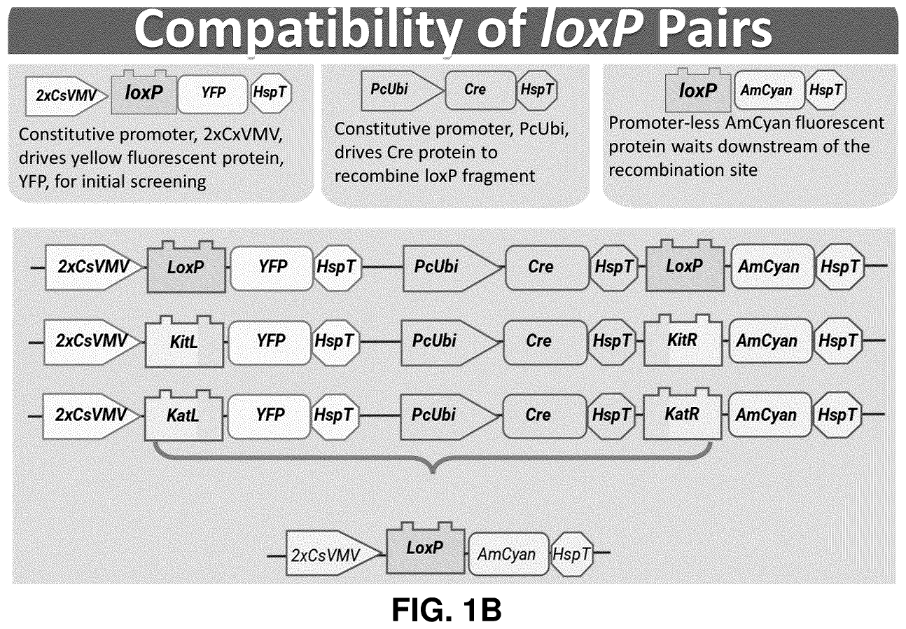

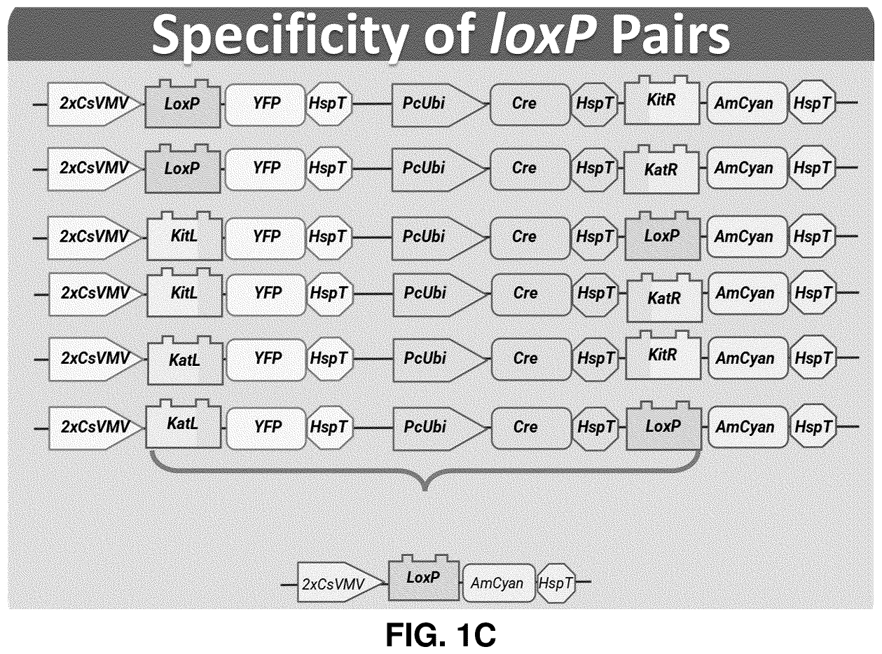

In some forms a “KitL” loxP sequence includes a first mutant left arm sequence and first mutant spacer. In some forms a “KitR” loxP sequence includes a first mutant right arm sequence and first mutant spacer. In some forms a “KitLR loxbox” sequence includes a first mutant left arm sequence and a first mutant right arm sequence, and a first mutant spacer (see FIG. 1A).

In some forms a “KatL” loxP sequence includes a second mutant left arm sequence and second mutant spacer. In some forms a “KatR” loxP sequence includes a second mutant right arm sequence and second mutant spacer. In some forms a “KatLR loxbox” sequence includes a second mutant left arm sequence and a second mutant right arm sequence, and a second mutant spacer (see FIG. 1A).

In some forms, the first and second mutants are orthogonal/non-equivalent, such that the first mutant left arm sequence does not recombine with the second mutant left arm sequence. The presence of different mutations in the first and/or second arm sequences can limit Cre's recognition of the mutant loxboxes (see FIGS. 1B and 1C).

i. Exemplary loxP Nucleotide Sequences

Exemplary wild-type and mutant loxP left arm region (lar), spacer (sp) and right arm region (rar) are provided.

In some forms, the wildtype loxP left arm region (lar) includes a nucleotide sequence of ATAACTTCGTATA (SEQ ID NO:19). In some forms, the wildtype loxP spacer (sp) includes a nucleotide sequence of GCATACAT (SEQ ID NO:20). In some forms, the wildtype loxP right arm region (rar) includes a nucleotide sequence of TATACGAAGTTAT (SEQ ID NO:21).

As used herein, the term “mutant loxP” refers to a nucleotide sequence corresponding to a loxP left arm region (lar) nucleotide sequence, or a loxP spacer (sp) nucleotide sequence, or a loxP right arm region (rar) nucleotide sequence that includes one or more nucleotide substitutions, additions or deletions relative to the corresponding wild-type loxP sequence(s).

In some forms, a mutant loxP left arm region includes a nucleotide sequence of cctAgTTCcTATA (SEQ ID NO:22; “double mutant 1LR lar”); a mutant spacer includes a nucleotide sequence of GCcTACAT (SEQ ID NO:23; “double mutant 1LR sp”); and mutant loxP right arm region includes a nucleotide sequence of TATAgGAtGgagg (SEQ ID NO:24; “double mutant 1LR rar”). In some forms, a mutant loxP spacer includes a nucleotide sequence of GGTTAACC (“CJ9”); or GGCTAGCC (“CJ10”); or GCGTACGC (“CJ11”); or GCCTAGGC (“CJ12”).

In some forms, a mutant loxP left arm region includes a nucleotide sequence of AgtAgaaCGTATA (SEQ ID NO:25; “double mutant 2LR lar”); a mutant spacer includes a nucleotide sequence of GgcTAatT (SEQ ID NO:26; “double mutant 2LR sp”); and mutant loxP right arm region includes a nucleotide sequence of TATACGAtGTagg (SEQ ID NO:27; “double mutant 2LR rar”).

In some forms, a mutant spacer includes a nucleotide sequence of GgcTAatT (SEQ ID NO: 28; “m3 sp”); or a mutant spacer includes a nucleotide sequence of ttcTAtcT (SEQ ID NO: 29; “m7 sp”).

In some forms, a mutant loxP left arm region includes a nucleotide sequence of taccgggCGTATA (SEQ ID NO:30; “lox75 lar”).

In some forms, a mutant loxP right arm region includes a nucleotide sequence of TATACGcccggta (SEQ ID NO:31; “KitR rar”).

In some forms, a mutant loxP left arm region (lar) includes a nucleotide sequence of AatgCaTgcTATA (SEQ ID NO:32; “lox44 lar”).

In some forms, a mutant loxP right arm region includes a nucleotide sequence of TATAgGTAccgAg (SEQ ID NO:33; “lox43 rar”).

In some forms, a mutant loxP left arm region includes a nucleotide sequence of taccgggCGTATA (SEQ ID NO:30; “KitL lar”); and a mutant spacer includes a nucleotide sequence of GCcTACAT (SEQ ID NO:35; “KitKat sp”).

In some forms, a mutant loxP left arm region includes a nucleotide sequence derived from Lox44, mutated to remove a “start” codon, having a sequence of AataCaTCGTATA (SEQ ID NO: 37; “KatL lar”).

In some forms, loxP mutant nucleotide sequences include a combinations of a left arm, a spacer and a right arm as set forth in any of SEQ ID NOs. 19-38.

In some forms, the loxP mutant nucleotide sequences include any one of SEQ ID NOs.3-14.

2. loxP-Cre Recombinase Mediated Exchange Cassette and Landing Pad

Engineered loxP-Cre mutants recombinase cassettes for iterative, targeted gene insertion in plant cells, plant tissues, plant materials, plant parts, and/or plants including compatible loxP pairs are provided.

Typically, the cassettes include a first promoter, an engineered mutant loxP sequence including a first mutant within one loxP “arm”, and optionally a first reporter gene.

In some forms the engineered loxP sequence including a first mutant within one loxP “arm” is a “KitL” loxP sequence, including a first mutant loxP left arm sequence.

In some forms the engineered loxP sequence including a first mutant within one loxP “arm” is a “KitR” loxP sequence, including a first mutant loxP right arm sequence.

In some forms the engineered loxP sequence including a first mutant within one loxP “arm” is a “KatL” loxP sequence, including a second mutant loxP left arm sequence.

In some forms the engineered loxP sequence including a first mutant within one loxP “arm” is a “KatR” loxP sequence includes a second mutant loxP right arm sequence.

In an exemplary form, a first recombinant DNA within the recipient genome cassette includes a first promoter (e.g., 2×CsVMV promoter), a first wild-type LoxP sequence, a KitR LoxP sequence, a first marker gene (e.g., YFP) that is driven by the first promoter, and ends transcription with a Heat Shock Protein terminator from Arabidopsis. Typically, the formation of the recombinant DNA within the recipient genome provides a distinct “Transcriptional unit” which includes each “promoter-terminator” sequence for each of the inserted genes, and/or for a specific marker.

In an exemplary form, a first recombinase mediated exchange cassette includes a second wild-type LoxP sequence, a KatR LoxP sequence, a second marker gene (e.g., AmCyan) that is promoter-less, but has a downstream Heat Shock Protein terminator followed by the first donor gene (e.g., Gain of Function 1; Goi1) and then the first KitL LoxP sequence. The promoter-less marker gene is configured such that it becomes operably linked to a promoter only after recombination, such that expression of the marker indicates successful recombination.

In some forms, recombination of the first cassette within the plant cell, plant tissue, plant material, plant part, and/or plant gives rise to formation of a second recombinant DNA within the recipient genome, including the first promoter (e.g., 2×CsVMV promoter), the second wild-type LoxP sequence, a KatR LoxP sequence, a second marker gene (e.g., AmCyan) that is driven by the 2×CsVMV promoter and terminated with the Heat Shock Protein terminator. The first donor gene (e.g., Gain of Function 1; “GOF1”/gene of interest 1; “Goi1”) is inserted into the recipient landing pad followed by the KitLR “loxbox” double mutant loxP sequence. The KitLR “loxbox” double mutant loxP sequence formed by the recombination of the KitL and KitR LoxP sequences is no longer tolerated by Cre and will give rise to no further recombination events.

In some forms, a second recombinase mediated exchange cassette includes a first wild-type LoxP sequence, a KitR LoxP sequence, a marker gene (e.g., YFP) that is driven by the first promoter only after recombination, and its transcription is terminator by the Heat Shock protein terminator a second donor gene (e.g., Gain of Function 2; Goi2) and a KatL LoxP sequence.

In some forms, a second recombinant DNA within the recipient genome results from recombination of the second recombinant DNA and second cassettes. In some forms, the second recombinant DNA includes a first promoter (e.g., 2×CsVMV promoter), a first wild-type LoxP sequence, a KitR LoxP sequence, a first marker gene (e.g., YFP) that is driven by the first promoter, and a first Heat Shock protein T gene, a second donor gene (e.g., Gain of Function 2; Goi2), a KatLR double mutant “loxbox” LoxP sequence, a second marker gene (e.g., AmCyan) that is promoter-less, a second Heat Shock protein T gene, a first donor gene (e.g., Gain of Function 1; Goi1); a KitLR “loxbox” double mutant loxP sequence, a first marker gene (e.g., YFP) that is driven by the first promoter, and a first Heat Shock protein T gene. The 2×CsVMV only drives the second YFP, the first YFP along with the AmCyan are promoterless and in theory are unable to be expressed, so the visual swap from AmCyan to YFP confirms recombination. In some forms a “KitLR loxbox” sequence includes a first mutant left arm sequence and a first mutant right arm sequence. In some forms a “KatLR loxbox” sequence includes a second mutant left arm sequence and a second mutant right arm sequence.

Schematics including each of the components of the described recombinase mediated exchange cassettes are illustrated in FIGS. 1A-1C.

i. Exemplary loxP-Cre Landing Pad in Target Plant Genome

As set forth in the Examples, step-wise insertion of one or more genes from a donor plant into a target recipient plant has been achieved the described RMEC compositions to achieve unidirectional step-wise iterative Cre-mediated recombination.

It has been established that the configuration/orientation of components within the recipient plant genome, and corresponding components within a loxP-Cre recombinase mediated exchange cassette (RMEC) including a gene of interest from a donor plant is significant for the process of providing recombinase mediated cassette exchange with enhanced efficiency within plants. RMCE including a wild-type loxP sequence upstream of a mutant loxP sequence can be inserted into a recipient plant genome for unidirectional gene insertion via RMCE in planta.

The described compositions and methods for RMCE inserts a gene of interest (GOI) from a donor plant into a recipient plant genome. Typically, the described compositions include the establishment of a first loxP “landing pad” sequence within a desired region within the recipient “target” plant genome.

Typically, the described first landing pad includes a wild-type (WT) loxP sequence upstream of and/or contiguous with a first mutant loxP sequence. Typically, a first mutant loxP sequence within the landing pad of the target plant genome is configured to be “compatible” with a second or further mutant loxP sequence within a recombination cassette. The compatibility between the first and second mutant loxP sequences includes a design that enables Cre-based recognition/recombination of each “single” mutant loxP but precludes Cre activity in the context of combining the two mutant loxP sequences aka the “loxbox”.

Typically, this system includes each mutant or a “pair” of mutant loxP sequences having a distinct mutation in either of the left arm region or right arm region of the loxP sequence relative to WT.

Exemplary “pairs” of loxP mutants that exhibit compatibility, i.e., which induce/permit Cre recombinase activity as a single mutant, but which will recombine to provide a “double” mutant loxP sequence that precludes Cre activity include:

Pair (1):
Lox75
(SEQ ID NO: 7)
and
Lox76;
(SEQ ID NO: 8)
Pair (ii):
Lox44
(SEQ ID NO: 9)
and
Lox43;
(SEQ ID NO: 10)
Pair (iii):
KitL
(SEQ ID NO: 11)
and
KitR;
(SEQ ID NO: 12)
Pair (iv):
KatL
(SEQ ID NO: 13)
and
KatR;
(SEQ ID NO: 14)

In some forms, the described landing pad and RMEC combinations for achieving RMCE in planta include one or more of the compatible loxP mutant pairs (i)-(iv), for example, whereby one member of a pair is present within the landing pad of a target plant genome and the compatible member of the pair is present within the RMEC containing a GOI for insertion into the target plant genome.

[symbol]

dtype loxP sequence contiguous with a first mutant loxP sequence, termed herein as a “landing pad”, includes a nucleic acid sequence configured according to the following General Formula I:

$$[ WT loxP ] - [ First mutant loxP ] \qquad ( Landing Pad 1 )$$

An exemplary WT loxP sequence includes the nucleic acid sequence

(SEQ ID NO: 2; “Wt LoxP”)
ATAACTTCGTATAGCATACATTATACGAAGTTAT.

An exemplary first mutant loxP sequence includes mutations within a left or right “arm” relative to the WT loxP sequence. An exemplary first mutant loxP sequence includes the nucleic acid sequence ATAACTTCGTATAGCcTACATTATACGcccggta (SEQ ID NO:12; “KitR”).

When the target genome is configured to receive more than a single GOI from one or more donors via more than a single integration step, the compositions and methods include establishing a first and second or further landing pads within the target plant genome.

In some forms, a landing pad has a nucleic acid sequence:

(SEQ ID NO: 15; Wt-KitR)
ATAACTTCGTATAGCATACATTATACGAAGTTATtaccgggCGTATAGC
cTACATTATACGAAGTTAT;

In some forms, a nucleotide sequence is incorporated between the WT and mutant loxP sequences. For example, in some forms, the landing pad components are separated by a nucleotide sequence from the backbone of a Greengate cloning plasmid: TGCTGGCCTT (SEQ ID NO: 34 “GP”).

For example, in some forms, a Wt-KitR landing pad including a spacer has a nucleic acid sequence:

(SEQ ID NO: 36; Wt-GP-KitR)
ATAACTTCGTATAGCATACATTATACGAAGTTATTGCTGGCCTTATAAC
TTCGTATAGCcTACATTATACGcccggta.

In some forms, a landing pad has a nucleic acid sequence:

(SEQ ID NO: 16; Wt-KitL)
ATAACTTCGTATAGCATACATTATACGAAGTTATATAACTTCGTATAGC
cTACATTATACGcccggta;

In some forms, a Wt-KitL landing pad including a GP spacer has a nucleic acid sequence:

(SEQ ID NO: 38; Wt-GP-KitL)
ATAACTTCGTATAGCATACATTATACGAAGTTATTGCTGGCCTTATAAC
TTCGTATAGCcTACATTATACGcccggta.

In some forms, a landing pad has a nucleic acid sequence:

(SEQ ID NO: 17; Wt-KatL)
ATAACTTCGTATAGCATACATTATACGAAGTTATAataCaTCGTATAGg
cTAatTTATACGAAGTTAT;

In some forms, a Wt-KatL landing pad including a GP spacer has a nucleic acid sequence:

(SEQ ID NO: 41; Wt-GP-KatL)
ATAACTTCGTATAGCATACATTATACGAAGTTATTGCTGGCCTTAataC
aTCGTATAGgcTAatTTATACGAAGTTAT.

In some forms, a landing pad has a nucleic acid sequence:

(SEQ ID NO: 18; Wt-KatR)
ATAACTTCGTATAGCATACATTATACGAAGTTATATAACTTCGTATAGg
cTAatTTATAgGTAccgAg;

in some forms, a Wt-KatR landing pad including a GP spacer has a nucleic acid sequence:

(SEQ ID NO: 42; Wt-GP-KatR)
ATAACTTCGTATAGCATACATTATACGAAGTTATTGCTGGCCTTATAAC
TTCGTATAGgcTAatTTATAgGTAccgAg.

ii. First Recombinase Mediated Exchange Cassette (RMEC)

In an exemplary form, a first RMEC includes a Wildtype (WT) loxP site, and two non-compatible mutant loxP sites (i.e., a second mutant loxP, and a third mutant loxP) flanking a gene of interest (GOI) from a donor plant.

In some forms, the first RMEC that includes one or more gene(s) of interest to be inserted into a “target” plant genome is configured to recognize the first “landing pad” within the target plant genome, i.e., configured according to General Formula I. Accordingly, the first RMEC is designed specifically to integrate the first gene(s) of interest (GOI-1) within the target plant genome in a user-defined/unidirectional manner.

In some forms, the described first RMEC nucleic acid sequence is configured according to the following General Formula II:

$$[ WT loxP ] - [ Second mutant loxP ] - [ GOI - 1 ] - [ Third mutant loxP ]$$

The first mutant loxP and third mutant loxP sequences must be a compatible pair (e.g., any one of pairs (i)-(iv)), and the second mutant loxP must be incompatible with the first and third mutant loxP sequences. For example, when the first and third mutant loxP sequences include the compatible pair (iii), the second mutant loxP sequence can be a member of pair (iv).

In some forms, the described loxP-Cre RMEC configured to integrate into the landing pad described above as a first integration step, includes a wildtype loxP sequence, and two non-compatible mutant loxP sequences that correspond to KatR+KitL, flanking DNA from a donor, i.e., including gene(s) to be inserted within a target plant.

For example, in some forms, a first RMEC sequence includes:

(SEQ ID NO: 2; “Wt LoxP”)
ATAACTTCGTATAGCATACATTATACGAAGTTAT;
(SEQ ID NO: 14; “KatR”)
ATAACTTCGTATAGgcTAatTTATAgGTAccgAg;
gene(s) of interest (GOI);
and
(SEQ ID NO: 11; “KitL”)
taccgggCGTATAGCcTACATTATACGAAGTTAT.

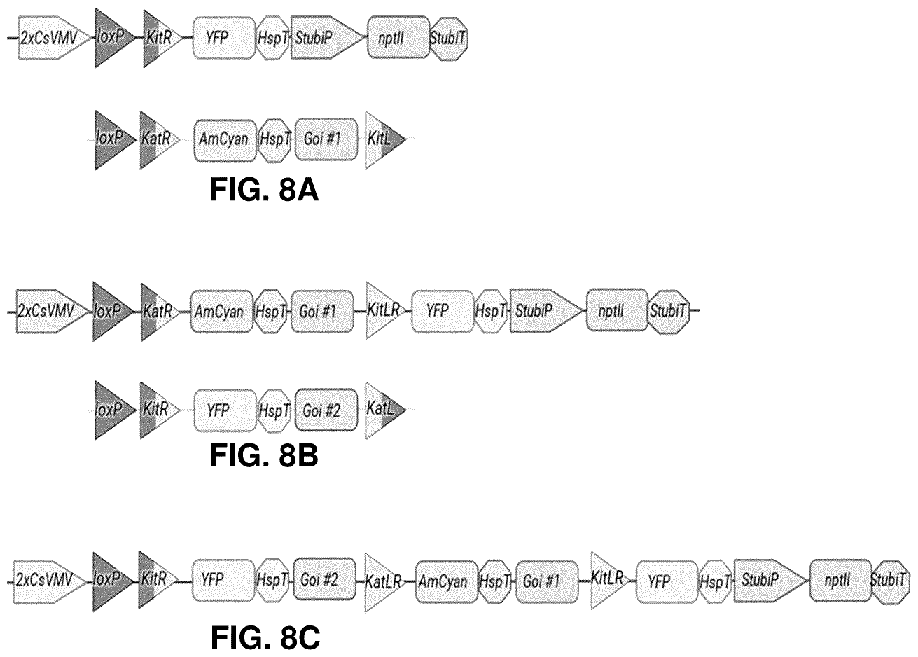

As set forth in FIGS. 8A-8B, the integration of the first RMEC via the landing pad region of the target genome results in:
(I) formation of a “landing pad 2” within the target genome, formed from the wildtype loxP sequence contiguous with the second mutant loxP sequence from the first RMEC; and
(II) formation of a “double-mutant” loxP sequence, which includes mutant loxP sequences from both the first mutant loxP within the first landing pad and the compatible third mutant loxP within the first RMEC, e.g., by combining the compatible loxP sequences of any one of pairs (i)-(iv).

The resulting “double-mutant” loxP sequence (termed herein as a “LoxBox”) precludes recognition/recombination via Cre. Accordingly, the formation of a first LoxBox by recombining the first and third mutant loxP sequences according to the described compositions for RMCE effectively removes the LoxP activity at the corresponding region within the target plant genome to preclude any further Cre-recombinase mediated events within this region of the target plant genome.

In some forms, a LoxBox has a nucleic acid sequence of:

(SEQ ID NO: 39; KitLKitR)
taccgggCGTATAGCcTACATTATACGcccggta;

In some forms, a LoxBox has a nucleic acid sequence of:

(SEQ ID NO: 40; KatLKatR)
AataCaTCGTATAGgcTAatTTATAgGTAccgAg.

In some forms, the first integration results in a nucleic acid sequence within the target plant genome that is configured according to the following General Formula III:

$${\underbrace{[ WT loxP ] - [ Second mutant loxP ]}}_{( Landing pad 2 )} - [ GOI - 1 ] - [ LoxBox ]$$

iii. Second Recombinase Mediated Exchange Cassette (RMEC)

In some forms, a second RMEC is configured to integrate into the landing pad of General Formula II (above) as a second integration step.

An exemplary second RMEC, configured to integrate into the new “landing pad 2” of General Formula III within the target plant genome includes a wildtype loxP sequence, a fourth mutant loxP sequence that is incompatible with the second mutant loxP sequence, a second gene of interest (GOI-2), and a fifth mutant loxP sequence that is compatible with the second mutant loxP sequence (i.e., forms a pair with the second mutant loxP sequence). In some forms, the described second RMEC nucleic acid sequence is configured according to the following General Formula IV:

$$[ WT loxP ] - [ Fifth mutant loxP ] - [ GOI - 2 ] - [ Fourth mutant loxP ]$$

The second mutant loxP and fourth mutant loxP sequences must be a compatible pair (e.g., any one of pairs (i)-(iv)), and the fifth mutant loxP must be incompatible with the third and fourth mutant loxP sequences. For example, when the second and fourth mutant loxP sequences include the compatible pair (vi), the second mutant loxP sequence can be a member of pair (iii).

In some forms, the described second RMEC configured to integrate into the landing pad of General Formula III as a second integration step, includes a wildtype loxP sequence, and two non-compatible mutant loxP sequences that correspond to KitR+KatL, flanking DNA from a second donor, i.e., including gene(s) to be inserted within a target plant.

For example, in some forms, a second RMEC sequence includes:

(SEQ ID NO: 2; “Wt LoxP”)
ATAACTTCGTATAGCATACATTATACGAAGTTAT;
(SEQ ID NO: 12; “KitR”)
ATAACTTCGTATAGCcTACATTATACGcccggta;
a second gene(s) of interest (GOI-2);
and
(SEQ ID NO: 13; “KatL”)
AataCaTCGTATAGgcTAatTTATACGAAGTTAT.

As set forth in FIGS. 8B-8C, the integration of the second RMEC via the landing pad region of the target genome results in:
(I) formation of a “landing pad 3” within the target genome, formed from the wildtype loxP sequence contiguous with the fifth mutant loxP sequence from the second RMEC; and
(II) formation of a second LoxBox sequence, which includes mutant loxP sequences from both the second mutant loxP within the landing pad 2 and the compatible fourth mutant loxP within the second RMEC, e.g., by combining the compatible loxP sequences of any one of pairs (i)-(iv).

The resulting “LoxBox 2” precludes recognition/recombination via Cre. Accordingly, the formation of a first and second LoxBoxes within the target plant genome according to the described compositions for RMCE effectively removes the LoxP activity at the corresponding regions within the target plant genome and precludes any further Cre-recombinase mediated recombination within these regions of the target plant genome.

[symbol] For example, in some forms, the second integration results in a nucleic acid sequence within the target plant genome that is configured according to the following General Formula V:

$$[ WT loxP ] - [ Fifth mutant loxP ] - [ GOI - 2 ] - [ loxBox 2 ] - [ GOI - 1 ] - [ Lox Box 1 ] \qquad ( Landing pad 3 )$$

It may be that the relative orientation of a WT and a mutant loxP sites within a recombination cassette and/or landing pad within a recipient genome influence the process of loxP-mediate recombination in planta. For example, in some forms, when a mutant loxP precedes a WT loxP, then the recombination would lead to “building” of genomic recombination sequences in the recipient plant genome in the opposite direction. Typically, the side of the landing pad with the mutant loxP is the side where the iterative insertions will “build”/elongate with in the recipient plant genome.

In some forms, the described compositions for loxP-Cre RMEC includes one or more reported genes. For example, in some forms, any one of the nucleic acid sequences set forth in General Formulas I-V include one or more reporter genes. For example, in some forms, any one of the nucleic acid sequences set forth in General Formulas I-V include at least one reporter gene contiguous with the one or more gene of interest. In some forms, the gene of interest is flanked by a reporter gene that is under the control of the same promoter as the gene of interest. In some forms, a reporter gene is a promoterless reporter gene. For example, in some forms, a reporter gene that is not operably linked to a promoter is contiguous with the gene(s) of interest within the recombination cassette, and becomes operably linked to a promoter in the recipient genome following successful recombination. An exemplary reporter gene is an AmCyan gene, or a YFP gene, or an antibiotic resistance gene.

a. Promoters

Plant promoters can be selected to control the expression of the transgene in different plant tissues or organelles, for all of which methods are known to those skilled in the art (Gasser, et al., Science, 244:1293-99 (1989)). The promoter can be a native plant or non-plant promoter or a synthetic promoter. The promoter can be constitutive, inducible, or tissue-specific. Examples of all of these types of promoters are known in the art can be utilized in the disclosed constructs, plants, and methods. See, e.g., Ali and Kim, A “Fruitful Decade Using Synthetic Promoters in the Improvement of Transgenic Plants,” Front. Plant Sci., 1 Nov. 2019, e.g., entire reference and Tables 1-3 therein, which is specifically incorporated by reference herein in its entirety.

Native plant promoters are typically more than 1000 bp long and are much weaker in their expression compared to constitutive viral promoters (e.g., CaMV 35S) that have been commonly exploited in plant biotechnology. In contrast, synthetic promoters are typically smaller in size but generate strong constitutive or inducible expression. The primary elements (e.g., core promoter and Cis-motifs) of different promoters can be linked together from diverse origins to form a synthetic promoter for the spatial and temporal control of a transgene in a genetically engineered plant. Different types of promoters are classified based on gene expression and their regulation. In constitutive expression, a promoter may be active throughout all developmental stages of the plant in each tissue. In contrast, an inducible promoter is modulated by external stimuli such as different biotic and abiotic environmental factors and/or at specific developmental stages without endogenous factors. Tissue-specific promoters direct the expression of a gene in one or more tissues or at certain stages of development.

In some embodiments, the promoter is a constitutive promoter of non-plant origin or plant origin. In some embodiments, the promoter is from a plant pathogen (such as cauliflower mosaic virus; CaMV 35S). Alternatively, constitutive promoters of plant origin include, but are not limited to, moderate constitutive promoter (PtMCP) isolated from Populus tomentosa; the parsley ubiquitin (PcUbi) promoter, PvUbi1 and PvUbi2 isolated from switchgrass; and CsCYP, C2 (CsGAPC2), and CsEF1 isolated from citrus.

In some embodiments, the promoter is an inducible promoter. Inducible promoters are frequently used because the gene of interest can be switched off or on under certain conditions and/or at certain developmental stages. Several different inducible promoters have been reported that vary according to their source and expression level. Inducible promoters can be grouped as either being chemically inducible (e.g., with steroids, metals, hormones, or alcohols, etc.), or physically inducible (e.g., with plant pathogens, temperature, light levels, stress, drought, dehydration).

A promoter whose activity is observed only in specific tissues of the plant is known as a tissue-specific promoter. In some embodiments, the promoter is tissue specific. Such promoters express the cistronic part of their gene in cells of particular tissue types, and their expression may also be induced in those tissues by internal or external factors. A transgene ligated to a tissue-specific promoter should activate expression of the encoded protein in a particular cell type, without affecting unmodified tissues of the plant.

In a preferred embodiment, promoters are selected from those of plant or prokaryotic origin that are known to yield high expression in plant nuclei or plastids. Promoters vary in their strength, i.e., ability to promote transcription. Depending upon the host cell system utilized, any one of a number of suitable promoters known in the art may be used. For example, for constitutive expression, the CaMV 35S promoter, the Arabidopsis actin promoter, or the soybean ubiquitin promoter may be used. For regulatable expression, the chemically inducible PR-1 promoter from tobacco or Arabidopsis may be used (see, e.g., U.S. Pat. No. 5,689,044 to Ryals, et al.).

In some embodiments, the promoter is drought or dehydration or other stress-induced promoter. Examples of such inducible promoters include, but are not limited to, Dhn4s, Dhn8s, HVA1s, Rab16Bj, wsi18j, HDZI-3, HDZI-4, Rab21, Wsi18, Lea3, Ugel, Dip1, and RIG1B (see, e.g., Xiao, et al., Yi Chuan, 28 (1): 85-91 (2006), Plant Biotechnol J, 18 (3): 829-844 (2000). doi: 10.1111/pbi.13252. Epub 2019 Sep. 25, Yi, et al., Planta, 232 (3): 743-54 (2010). doi: 10.1007/s00425-010-1212-z. Epub 2010 Jun. 22, Zhu, et al., Yi Chuan, 32 (3): 229-34 (2010). doi: 10.3724/sp.j.1005.2010.00229, and Rahamkulov, et al., 3 Biotech, 10 (10): 426 (2020). doi: 10.1007/s13205-020-02350-x. Epub 2020 Sep. 11 each of which is specifically incorporated by reference herein in its entirety). Another suitable category of promoters is wound inducible. Numerous promoters have been described which are expressed at wound sites. Preferred promoters of this kind include those described by Stanford, et al., Mol. Gen. Genet. 215:200-208 (1989), Xu et al. Plant Molec. Biol. 22:573-588 (1993), Logemann, et al., Plant Cell 1:151-158 (1989), Rohrmeier, et al., Plant Molec. Biol. 22:783-792 (1993), Firek, et al., Plant Molec. Biol. 22:129-142 (1993), and Warner, et al., Plant J., 3:191-201 (1993).

In some forms, the promoter is a plant expressible promoter, such as a constitutive promoter, i.e. a promoter capable of directing high levels of expression in most cell types (in a spatio-temporal independent manner). Examples of plant expressible constitutive promoters include promoters of bacterial origin, such as the octopine synthase (OCS) and nopaline synthase (NOS) promoters from Agrobacterium, but also promoters of viral origin, such as that of the cauliflower mosaic virus (CaMV) 35S transcript (Hapster et al., 1988, Mol. Gen. Genet. 212: 182-190) or 19S RNAs genes (Odell et al., 1985, Nature. 6; 313 (6005): 810-2; U.S. Pat. No. 5,352,605; WO 84/02913; Benfey et al., 1989, EMBO J. 8:2195-2202), the enhanced 2×35S promoter (Kay at al., 1987, Science 236:1299-1302; Datla et al. (1993), Plant Sci 94:139-149) promoters of the cassava vein mosaic virus (CsVMV; WO 97/48819, U.S. Pat. No. 7,053,205), 2×CsVMV (WO2004/053135) the circovirus (AU 689 31 1) promoter, the sugarcane bacilliform badnavirus (ScBV) promoter (Samac et al., 2004, Transgenic Res. 13 (4): 349-61), the figwort mosaic virus (FMV) promoter (Sanger et al., 1990, Plant Mol Biol. 14 (3): 433 3), the subterranean clover virus promoter No 4 or No 7 (WO 96/06932) and the enhanced 35S promoter as described in US 5, 164,316, U.S. Pat. Nos. 5,196,525, 5,322,938, 5,359,142 and 5,424,200. Among the promoters of plant origin, mention will be made of the promoters of the plant ribulose-biscarboxylase/oxygenase (Rubisco) small subunit promoter (U.S. Pat. No. 4,962,028; WO99/25842) from Zea mays and sunflower, the promoter of the Arabidopsis thaliana histone H4 gene (Chaboute et al., 1987), the ubiquitin promoters (Holtorf et al., 1995, Plant Mol. Biol. 29:637-649, U.S. Pat. No. 5,510,474) of Maize, Rice and sugarcane, the Rice actin 1 promoter (Act-1, U.S. Pat. No. 5,641,876), the histone promoters as described in EP 0 507 698 A1, the Maize alcohol dehydrogenase 1 promoter (Adh-1) (from http://www.patentlens.net/daisy/promoters/242.html)). Also the small subunit promoter from Chrysanthemum may be used if that use is combined with the use of the respective terminator (Outchkourov et al., Planta, 216:1003-1012, 2003).

Suitable tissue specific expression patterns include green tissue specific, root specific, stem specific, and flower specific. Promoters suitable for expression in green tissue include many which regulate genes involved in photosynthesis, and many of these have been cloned from both monocotyledons and dicotyledons. A suitable promoter is the maize PEPC promoter from the phosphoenol carboxylase gene (Hudspeth, et al., Plant Molec. Biol. 12:579-589 (1989)). A suitable promoter for root specific expression is that described by de Framond, FEBS, 290:103-106 (1991); EP 0 452 269 to de Framond and a root-specific promoter is that from the T-1 gene. A suitable stem specific promoter is that described in U.S. Pat. No. 5,625,136 and which drives expression of the maize trpA gene.

Although in some embodiments, strong promoters are preferred, in other embodiments, the promoter can be a relatively weak plant expressible promoter. Thus, the promoter can in some embodiments initiate and control transcription of the operably linked nucleic acids about 10 to about 100 times less efficient that an optimal CaMV35S promoter. Relatively weak plant expressible promoters include the promoters or promoter regions from the opine synthase genes of Agrobacterium spp. Such as the promoter or promoter region of the nopaline synthase, the promoter or promoter region of the octopine synthase, the promoter or promoter region of the mannopine synthase, the promoter or promoter region of the agropine synthase and any plant expressible promoter with comparably activity in transcription initiation. Other relatively weak plant expressible promoters may be dehiscence zone selective promoters, or promoters expressed predominantly or selectively in dehiscence zone and/or valve margins of fruits, such as the promoters described in WO97/13865.

Cis-regulatory elements from the promoter of photoperiod-responsive genes, coordinated motifs integrating hormones and stresses to photoperiod responses, and the promoters of photo-responsive genes such as those described in Mongkolsiriwatana, Katsetsart J. (Nat. Sci.) 43:164-177 (2009), can also be used.

In some forms, a first or second promoter is a 2×CsVMV promoter. In some forms, a first or second promoter is a PcUbi promoter.

b. Selectable Markers/Reporter Genes

Genetic constructs may encode a selectable marker to enable selection of transformation events, e.g., nuclear transformation events. There are many methods that have been described for the selection of transformed plants [for review see (Miki et al., Journal of Biotechnology, 107:193-232 (2004) and references incorporated within]. Selectable marker genes that have been used extensively in plants include the neomycin phosphotransferase gene nptII (U.S. Pat. Nos. 5,034,322, 5,530,196), hygromycin resistance gene (U.S. Pat. No. 5,668,298), the bar gene encoding resistance to phosphinothricin (U.S. Pat. No. 5,276,268), the expression of aminoglycoside 3″-adenyltransferase (aadA) to confer spectinomycin resistance (U.S. Pat. No. 5,073,675), the use of inhibition resistant 5-enolpyruvyl-3-phosphoshikimate synthetase (U.S. Pat. No. 4,535,060) and methods for producing glyphosate tolerant plants (U.S. Pat. Nos. 5,463,175; 7,045,684).

Methods of plant selection that do not use antibiotics or herbicides as a selective agent have been previously described and include expression of glucosamine-6-phosphate deaminase to inactive glucosamine in plant selection medium (U.S. Pat. No. 6,444,878) and a positive/negative system that utilizes D-amino acids (Erikson, et al., Nat Biotechnol, 22:455-8 (2004). European Patent Publication No. EP 0 530 129 A1 describes a positive selection system which enables the transformed plants to outgrow the non-transformed lines by expressing a transgene encoding an enzyme that activates an inactive compound added to the growth media. U.S. Pat. No. 5,767,378 describes the use of mannose or xylose for the positive selection of engineered plants. Methods for positive selection using sorbitol dehydrogenase to convert sorbitol to fructose for plant growth have also been described (WO 2010/102293). Screenable marker genes include the beta-glucuronidase gene (Jefferson, et al., EMBO J. 6:3901-3907 (1987); U.S. Pat. No. 5,268,463) and native or modified green fluorescent protein gene (Cubitt, et al., Trends Biochem. Sci. 20:448-455 (1995); Pan, et al., Plant Physiol. 112:893-900 (1996)).

Transformation events can also be selected through visualization of fluorescent proteins such as the fluorescent proteins from the nonbioluminescent Anthozoa species which include DsRed, a red fluorescent protein from the Discosoma genus of coral (Matz, et al., Nat Biotechnol, 17:969-73 (1999)). An improved version of the DsRed protein has been developed (Bevis, et al., Nat Biotech, 20:83-87 (2002)) for reducing aggregation of the protein. Visual selection can also be performed with the yellow fluorescent proteins (YFP) including the variant with accelerated maturation of the signal (Nagai, et al., Nat Biotech, 20:87-90 (2002), the blue fluorescent protein, the cyan fluorescent protein, and the green fluorescent protein (Sheen, et al., Plant J, 8:777-84 (1995); Davis, et al., Plant Molecular Biology, 36:521-528 (1998)). A summary of fluorescent proteins can be found in Tzfira, et al., Plant Molecular Biology, 57:503-516 (2005) and Verkhusha, et al., Nat Biotech, 22:289-296 (2004) whose references are incorporated in their entirety. Improved versions of many of the fluorescent proteins have been made for various applications. Use of the improved versions of these proteins or the use of combinations of these proteins for selection of transformants will be obvious to those skilled in the art. It is also practical to simply analyze progeny from transformation events for the presence of the PHB thereby avoiding the use of any selectable marker.

For plastid transformation constructs, a preferred selectable marker is the spectinomycin-resistant allele of the plastid 16S ribosomal RNA gene (Staub, et al., Plant Cell 4:39-45 (1992); Svab, et al., Proc. Natl. Acad. Sci. USA 87:8526-8530 (1990)). Selectable markers that have since been successfully used in plastid transformation include the bacterial aadA gene that encodes aminoglycoside 3′-adenyltransferase (AadA) conferring spectinomycin and streptomycin resistance (Svab, et al., Proc. Natl. Acad. Sci. USA, 90:913-917 (1993), nptII that encodes aminoglycoside phosphotransferase for selection on kanamycin (Carrer, et al., Mol. Gen. Genet. 241:49-56 (1993); Lutz, et al., Plant J., 37:906-913 (2004); Lutz K A, et al., Plant Physiol. 145:1201-1210 (2007)), aphA6, another aminoglycoside phosphotransferase (Huang F-C, et al, Mol. Genet. Genomics 268:19-27 (2002)), and chloramphenicol acetyltransferase (Li, et al., Plant Mol Biol, DOI 10.1007/s11103-010-9678-4 (2010)). Another selection scheme has been reported that uses a chimeric betaine aldehyde dehydrogenase gene (BADH) capable of converting toxic betaine aldehyde to nontoxic glycine betaine (Daniell, et al., Curr. Genet., 39:109-116 (2001)).

In some forms, a first or second marker is a yellow fluorescent protein, YFP gene. In some forms, a first or second marker is a AmCyan fluorescent protein gene.

c. Target Plant Cell, Plant Tissue, Plant Material, Plant Part, and/or Plant

The disclosed compositions for genetic recombination can be transformed into any suitable plant cell, plant tissue, plant material, plant part, and/or plant to provide an engineered plant cell, plant tissue, plant material, plant part, and/or plant expressing a donor gene.

Both dicotyledons (“dicots”) and monocotyledons (“monocots”) can be used as sources. Monocot seedlings typically have one cotyledon (seed-leaf), in contrast to the two cotyledons typical of dicots. Eudicots are dicots whose pollen has three apertures (i.e. triaperturate pollen), through one of which the pollen tube emerges during pollination. Eudicots contrast with the so-called ‘primitive’ dicots, such as the magnolia family, which have uniaperturate pollen (i.e. with a single aperture).

Monocots include one of the large divisions of Angiosperm plants (flowering plants with seeds protected within a vessel). They are herbaceous plants with parallel veined leaves and have an embryo with a single cotyledon, as opposed to dicot plants (dicotyledonous), which have an embryo with two cotyledons. Most of the important staple crops of the world, the so-called cereals, such as wheat, barley, rice, maize, sorghum, oats, rye and millet, are monocots. Thus, the plant cell, plant tissue, plant material, plant part, and/or plant can be a grass, such as wheat, barley, rice, maize, sorghum, oats, rye and millet.

The plant cell, plant tissue, plant material, plant part, and/or plant can therefore be a cereal crop such as wheat, oat, barley, or rice; a forage such as bahiagrass, dallisgrass, kleingrass, guineagrass, reed canarygrass, orchardgrass, ricegrass, foxtail, or vetch; a legume such as soybean, lentil, or chickpea; an oilseed such as canola; a vegetable such as onion or carrot; or a specialty crop such as caraway, hemp, or sesame.

Examples of wheat include, but are not limited to, T. aestivum, T. aethiopicum, T. araraticum, T. boeoticum, T. carthlicum, T. compactum, T. dicoccoides, T. dicoccon, T. durum, T. ispahanicum, T. karamyschevii, T. monococcum, T. polonicum, T. spelta, T. thaoudar, T. timopheevii, T. turanicum, T. turgidum, T. urartu, T. vavilovii, and T. zhukovskyi.

In some embodiments, the plant cell, plant tissue, plant material, plant part, and/or plant is alfalfa, i.e., Medicago sativa.

In some embodiments, the plant cell, plant tissue, plant material, plant part, and/or plant is a soybean. For example, the soybean plant cell, plant tissue, plant material, plant part, and/or plant can be Jack, Resnik, Williams 82, Corsoy, Crawford, Hutcheson, Kunitz, Champ, Benning, or Woodruff. Additional suitable soybean varieties are available from both academic and commercial institutions, such as—for example—the University of Guelph (Ontario Agricultural College; e.g. soybean varieties RCAT Staples, Westag 97, RCAT Bobcat, OAC Prudence, OAC Woodstock, OAC 9908), or soybean varieties from Daryland or Soygenetics. Additional suitable varieties are P1548402 (Peking), P1437654 (Er-hejjan), P1438489 (Chiquita), P1507354 (Tokei 421), P1548655 (Forrest), P1548988 (Pickett), P188788, P1404198 (Sun Huan Do), P1404166 (Krasnoaarmejkaja), Hartwig, Manokin, Doles, Dyer, and Custer.

In some embodiments, the plant cell, plant tissue, plant material, plant part, and/or plant is a miscanthus. Thus, the plant cell, plant tissue, plant material, plant part, and/or plant can be of the species Miscanthus floridulus, Miscanthus×. giganteus, Miscanthus sacchariflorus (Amur silver-grass), Miscanthus sinensis, Miscanthus tinctorius, or Miscanthus transmorrisonensis.

In some embodiments, the plant cell, plant tissue, plant material, plant part, and/or plant is a Brassicaceae family member, including but not limited to those mentioned above.

Additional representative plant cell, plant tissue, plant material, plant part, and/or plant useful in the compositions and methods disclosed herein include tomatoes; poplar; crops harvested as biomass, such as silage corn, alfalfa, switchgrass, and tobacco; industrial oilseeds such as Camelina sativa, Crambe, Jatropha, castor; cottonseed; sunflower; palm; coconut; rice; safflower; peanut; mustards including Sinapis alba, sugarcane and flax.

### III. Methods for Recombinase Mediated Cassette Exchange

Methods for Recombinase Mediated Cassette Exchange (RMCE) in plants are also provided.

Typically, the methods introduce one or more user-defined genes at a user defined location within a plant cell, plant tissue, plant material, plant part, and/or plant genome in a monitored manner.

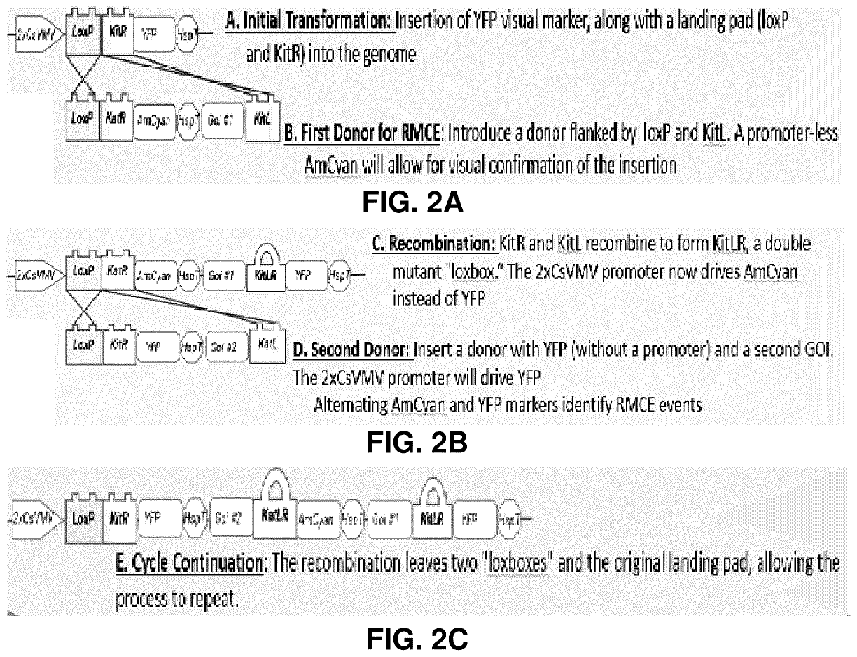

The methods generally include step-wise manipulation of a target genome within a target plant cell, plant tissue, plant material, plant part, and/or plant for insertion of a target gene from a donor plant cell, plant tissue, plant material, plant part, and/or plant. In some forms, the method steps include a first step to insert a first loxP “arm” mutant landing pad into a target plant cell, plant tissue, plant material, plant part, and/or plant genome at a user-defined location together with a first marker; a second step to introduce a donor gene using an opposing “arm” mutant loxP sequence together with a second (e.g., promoter less) marker; a third, “recombination” step to recombine the two loxP regions and form a first double mutant “loxbox”; a fourth step to introduce a second donor gene using an opposing “arm” mutant loxP sequence together with a third promoter-driven marker; and a fifth “recombination” step to recombine the second set of two loxP regions and form a second double mutant “loxbox” (see FIGS. 2A-2C; 8A-8C).

In some forms, the process for recombinase-mediated cassette exchange (RMCE) includes:
(a) a first initial transformation step of inserting a marker gene (e.g., YFP visual marker) along with a “landing pad” (loxP with KitR sequence) into a target plant cell, plant tissue, plant material, plant part, and/or plant genome.

In some forms, the process for recombinase-mediated cassette exchange includes:
(b) a second transformation step to introduce a “donor for RMCE” construct, including a gene flanked by a loxP with a KitL sequence. In some forms, a promoter-less AmCyan gene is also included, to allow for visual confirmation of the insertion.

In some forms, the process for recombinase-mediated cassette exchange includes:
(c) a third, recombination step, whereby the KitR and KitL recombine to form KitLR, a double mutant “loxbox”. The 2×CsVMV promoter now drives the AmCyan gene instead of the YFP gene. The KitLR “loxbox” double mutant loxP sequence formed by the recombination of the KitL and KitR LoxP sequences is no longer tolerated by Cre and will give rise to no further recombination events.

In some forms, the process for recombinase-mediated cassette exchange includes:
(d) a fourth step of transformation to introduce a “second donor for RMCE” construct. In some forms, the fourth step inserts a donor with YFP (without a promoter) and a second GOI.

In some forms, the process for recombinase-mediated cassette exchange includes:
(e) a fifth step of recombination between the second donor for RMCE″ construct to form a fifth recombinase mediated exchange cassette, including a first promoter (e.g., 2×CsVMV promoter), and a first wild-type LoxP sequence, together with a KitR LoxP sequence, a first marker gene (e.g., YFP) that is driven by the first promoter, and a first Heat Shock protein T gene, a second donor gene (e.g., Gain of Function 2; Goi2), a KatLR double mutant “loxbox” LoxP sequence, a second marker gene (e.g., AmCyan) that is promoter-less, a second Heat Shock protein T gene, a first donor gene (e.g., Gain of Function 1; Goi1); a KitLR “loxbox” double mutant loxP sequence, a first marker gene (e.g., YFP) that is driven by the first promoter, and a first Heat Shock protein T gene. The 2×CsVMV promoter will drive YFP.

In some forms, alternating AmCyan and YFP markers identify RMCE events.

According to the described methods, the process for recombinase-mediated cassette exchange produces two double mutant loxP sequence “loxboxes” that are no longer able to engage Cre recombination, together with a single loxP arm mutant “landing pad” sequence. The presence of the loxP arm mutant “landing pad” sequence enables for the repeatable introduction of a third or further “gain-of function” donor gene in the same region.

In some forms, the process for recombinase-mediated cassette exchange optionally includes a sixth or further step of
(f) repeating any of steps (a) to (e) to introduce one or more further gain of function genes into the same location in the target plant cell, plant tissue, plant material, plant part, and/or plant genome.

The subsequent addition of further donor gene(s) within the same region is monitored in the same manner, e.g., by the sequential visualization of promoter-driven, or promoter-less markers, respectively, allowing the process to repeat through as many iterations as required using the same cassette framework (see FIGS. 2A-2C).

An exemplary system for application of the described methods includes:
(i) recombinase-mediated cassette exchange in a target plant cell, plant tissue, plant material, plant part, and/or plant, as described in steps (a)-(f); and
(ii) analysis of recombination in the target plant cell, plant tissue, plant material, plant part, and/or plant.

An exemplary methodology for the step of analysis of recombination in the target plant cell, plant tissue, plant material, plant part, and/or plant includes one or more of
(I) biopsy of one or more parts of the target plant (such as a leaf or shoot) at a time appropriate for the expression and detection of a first and/or second detectable marker in the parts of the target plant cell, plant tissue, plant material, plant part, and/or plant;
(II) Samples homogenized and aliquoted for 5 reads per biopsy & 4 biopsies per plant cells(s) and/or part (e.g., leaf);
(III) fluorescent quantification (e.g., using Synergy H1 Reader); and
(IV) Determining recombination rate (e.g., as a function fop AmCyan/YFP).

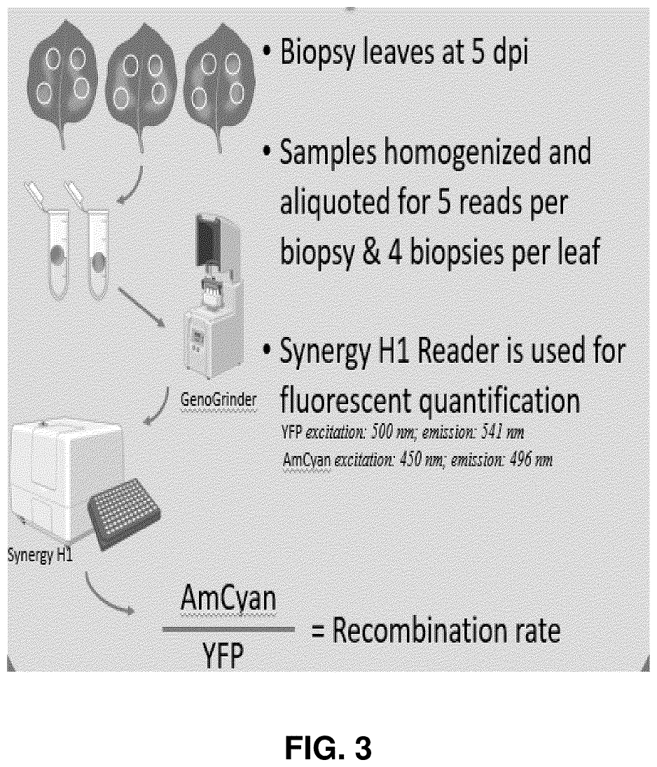

An exemplary workflow is depicted in FIG. 3.

The described stepwise and unidirectional manner of integration provides flexibility to develop custom-made gene stacks and permits additional genes to be added to meet future needs.

Methods for enhanced loxP-Cre recombinase mediated cassette exchange (RMCE) of one or more gene(s) of interest from a donor plant to a target plant have been developed.

In an exemplary workflow for iterative RMCE-mediated gene exchange, the methods include step-wise insertions of two or more genes from the same or different donor plant(s) into a user defined region of a target/recipient plant genome. The methods use the described RMEC compositions to achieve unidirectional Cre-mediated recombination to
(i) insert a first loxP “landing pad” sequence (including a wild-type (WT) loxP sequence contiguous with a first mutant loxP sequence) within a desired region within the recipient “target” plant genome;
(ii) performing a first iteration of Cre-recombinase mediated RMCE by contacting the first landing pad with a first recombination mediated exchange cassette (RMEC) including a third (compatible) mutant loxP sequence and a wild-type loxP sequence flanking a first gene of interest,
whereby the first gene of interest is inserted by RMCE into the target plant genome,
whereby the third mutant loxP sequence is recombined with the first mutant loxP sequence to form a non-functional first LoxBox sequence in the target plant genome, and
whereby the RMEC also includes a second mutant loxP sequence that is non-compatible with the first or third mutant loxP sequences, and wherein the second mutant loxP sequence is configured to form, together with the wild-type loxP sequence, a second landing pad in the target genome;
(iii) optionally verifying Cre-mediated recombination following the first iteration of RMCE;
(iv) performing a second iteration of Cre-recombinase mediated RMCE by contacting the second landing pad with a second recombination mediated exchange cassette (RMEC) including a fourth mutant loxP sequence that is compatible with the second mutant loxP sequence in the second landing pad, and a wild-type loxP sequence flanking a second gene of interest,
whereby the second gene of interest is inserted by RMCE into the target plant genome,
whereby the fourth mutant loxP sequence is recombined with the second mutant loxP sequence to form a non-functional second LoxBox sequence in the target plant genome, and
whereby the RMEC also includes a fifth mutant loxP sequence that is non-compatible with the second or fourth mutant loxP sequences, and wherein the fifth mutant loxP sequence is configured to form, together with the wild-type loxP sequence, a third landing pad in the target genome; and
(v) optionally verifying Cre-mediated recombination following the second iteration of RMCE.

In some forms, the methods repeat steps (i)-(iv) one or more times to insert a multiplicity of genes of interest into a target plant genome.

### A. Methods for Reproducing Transgenic Plants

Following transformation by any one of the methods described above, the following procedures can be used to obtain a transformed plant expressing the transgenes: select the plant cells that have been transformed on a selective medium; regenerate the plant cells that have been transformed to produce differentiated plants; and optionally select transformed plants expressing the transgene producing the desired level of desired polypeptide(s) in the desired tissue and cellular location. In some forms, the recombination occurs in a benthi plant. Typically, when the methods include a benthi recipient plant, selection is carried out visually.

Methods can include vegetative propagation, e.g., for forest/fruit/nut trees or species that do not produce seed. Vegetative propagation is an asexual method of plant reproduction that occurs in its leaves, roots and stem. This can occur through fragmentation and regeneration of specific vegetative parts of plants. Vegetative propagation can be naturally e.g., without further human intervention, or artificially, e.g., by cutting, grafting, layering, tissue culturing, etc. In some forms, for plastid transformation procedures, further rounds of regeneration of plants from explants of a transformed plant or tissue can be performed to increase the number of transgenic plastids such that the transformed plant reaches a state of homoplasmy (all plastids contain uniform plastomes containing transgene insert). The cells that have been transformed may be grown into plants in accordance with conventional techniques. See, for example, McCormick et al. Plant Cell Reports, 5:81-84 (1986). These plants may then be grown, and either pollinated with the same transformed variety or different varieties, and the resulting hybrid having constitutive expression of the desired phenotypic characteristic identified. Two or more generations may be grown to ensure that constitutive expression of the desired phenotypic characteristic is stably maintained and inherited and then seeds harvested to ensure constitutive expression of the desired phenotypic characteristic has been achieved. In some scenarios, it may be advantageous to insert a multi-gene pathway into the plant by crossing of lines containing portions of the pathway to produce hybrid plants in which the entire pathway has been reconstructed. This is especially the case when high levels of product in a seed compromises the ability of the seed to germinate or the resulting seedling to survive under normal soil growth conditions. Use of lines that possess cytoplasmic male sterility (Esser, et al., Progress in Botany, Springer Berlin Heidelberg. 67:31-52 (2006)) with the appropriate maintainer and restorer lines allows these hybrid lines to be produced efficiently. Cytoplasmic male sterility systems are already available for some Brassicaceae species (Esser, et al., Progress in Botany, Springer Berlin Heidelberg. 67:31-52 (2006)).

The engineered plant cells, plant tissues, plant materials, plant parts, and/or plants, most particularly, but not limited to, the transgenic plants, can be breed with themselves, or with other plants to create hybrid plants. The plants disclosed herein include hybrid plants which can be produced using any known breeding techniques. Hybrids are the product of a cross between genetically different parents. The development of hybrids in a plant breeding program often involves the development of homozygous inbred lines, the crossing of these lines, and the evaluation of the crosses. Most plant breeding programs combine the genetic backgrounds from two or more inbred lines or various other broad-based sources into breeding pools from which new inbred lines are developed by selfing and selection of desired phenotypes. Hybrids can also be used as a source of plant breeding material or as source populations from which to develop or derive new plant lines. The expression of a trait in a hybrid may exceed the midpoint of the amount expressed by the two parents, which is known as hybrid vigor. Methods for evaluating the presence of donor characteristics can be accomplished using any technique known in the art. Because quantitative traits are phenotypic characteristics that vary in degree and may include environmental influence, breeders may also take into consideration the breeding environment and or breeding location when cultivating the plants disclosed herein.

### IV. Methods of Use

Methods of using the disclosed plant cells, plant tissues, plant materials, plant parts, and/or plants are also provided and include planting an engineered plant such as a transgenic plant, or a part thereof such as seeds, optionally growing the plant for a fixed or indefinite period of time, and optionally harvesting the plant or part(s) thereof.

In some embodiments, the engineered plant cells, plant tissues, plant materials, plant parts, and/or plants produced according to the disclosed methods are particularly resilient in tissue culture and in soil. In some embodiments, the engineered plants are well suited to withstand water- and nutrient-deficient conditions for extended periods of time relative to their wildtype counterparts. In some embodiments, the engineered plants can overcome natural (e.g., environmental) or induced (e.g., at the hand of man) growth penalties including, but not limited to, juvenile senescence, dwarfism, temperature-sensitive dwarfism, and/or the like. In some embodiments, the engineered plants highly suited for improving crop growth including, but not limited to, forage or bioenergy crop growth, in marginal land.

### EXAMPLES

### Example 1: Development of LOXBOX System for Gene Stacking in Planta

Systems and methods for genetic modification of a plant, including at least one mutant loxP sequence including a left arm region and a right arm region and a spacer sequence, have been developed and tested. The systems use the Cre-Lox recombinase system for unidirectional, predictable, targeted, iterative gene insertion for gene stacking, and effectively circumvent the retention of superfluous loxPs post-recombination.

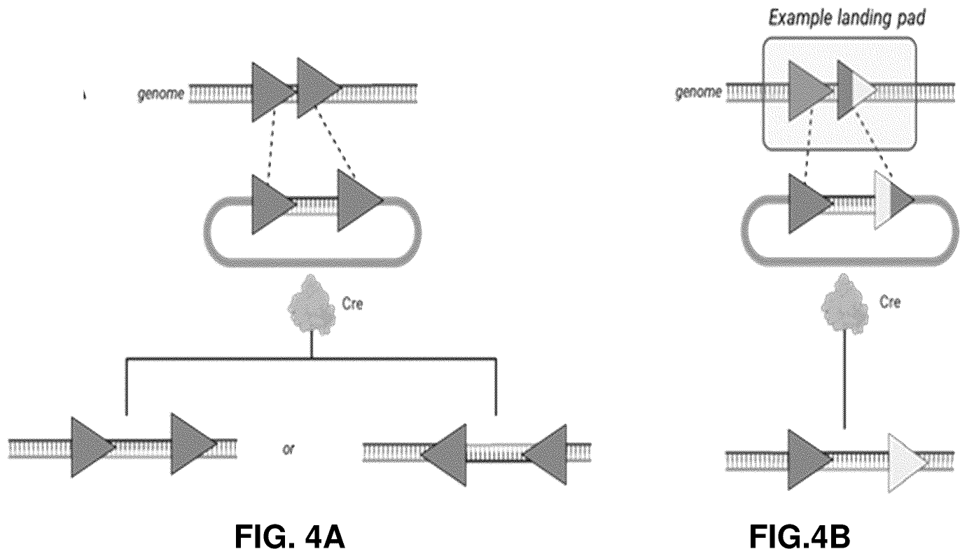

First, the process must ensure the directionality of insertions so that integrations are predictable and genes can stack in one direction without interfering with previous insertions. Using two recombinase sites to flank genes of interest is referred to as recombinase mediated cassette exchange (RMCE). If only wtloxP sites are used, the flanked gene could be inserted either in the forward or reverse direction. However, loxP pairs with different spacers were testing so they can only recombine with their respective partners, which would force the reaction to only insert in the forward direction, not allowing for the reverse. The term “landing pad” specifically refers to the combination of two loxP sites with different spacers, a wtloxP followed by a mutant (FIGS. 4A-4B). Furthermore, in addition to using different spacers from the wtloxP, using sites with mutated arms would lead to the development of a “Loxbox” that would prevent further insertions at that site. This would however require a new loxP site for further RMCE.

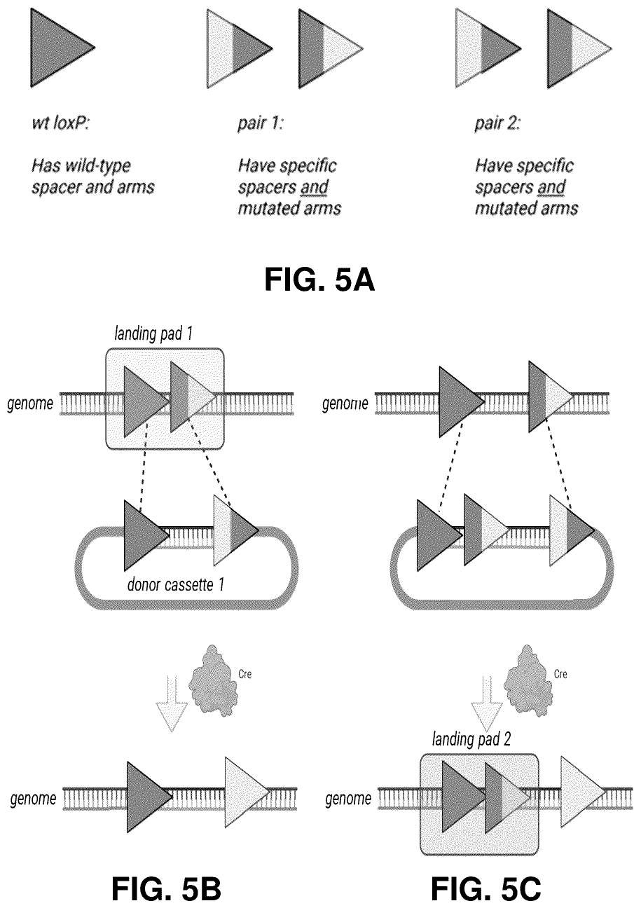

Second, a method must allow for the insertion of a new gene and a new targetable region, aka landing pad, for the next insertion. To enable further insertions, a new loxP mutant could be placed in the donor to allow it to form the next landing pad (FIGS. 5A-5B). This site would have to be unable to recombine with either of the other pairs.

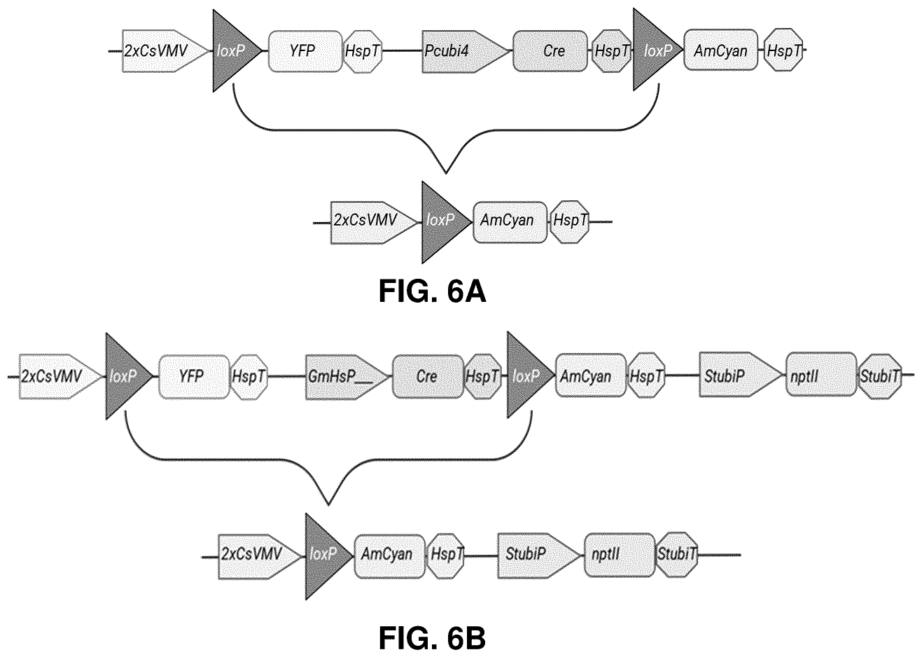

The third goal for developing iterative targetable RMCE is to have bespoke recombinase sites that work in pairs without crosstalk between the pairs must be developed and utilized. Since only pairs of sites with matching spacers can recombine (Missirlis et al., 2006), the landing pad should consist of two recombinase sites with differing spacers to ensure the gene is inserted in only one direction. This way, the inserted gene will still be flanked by recombinase sites, and if there is no crosstalk between the pairs, a third recombinase site can be introduced via the insert. To inactivate the downstream recombinase site and prevent ejection of the insert, mutated arms should create recombinants that cannot be recognized by Cre after recombination. With this system, each integration of an insert includes the landing pad for the next insertion while rendering the downstream recombinase site unrecognizable to prevent unintended ejections (FIGS. 6A-6C).

### Methods

The proposed landing pad design is inspired by Zhu et al.'s (2017a) approach to gene stacking in bacteria. By leveraging the specificity of loxP spacers and the lack of recognition by Cre towards double mutant arms, combinations of loxP-mutant recombinase sites can be utilized to ensure unidirectional recombinase-based cassette exchange with predictable directionality.

To establish a landing pad in planta, it is essential to confirm that all elements are functional. This includes not only the custom-designed loxP sites but also a reliable inducible promoter to prevent unintended expression and recombination.

The new loxP sites (shown in Table 1) incorporate spacers. To ensure obtaining specific loxP pairs, spacers which demonstrated specificity in Eukaryotes when compared directly with one another and with the wtloxP sequence are included. Their arms are derived from lox75, lox76, lox43, and lox44 (Albert et al., 1995), which have been validated for functionality and 5 exhibit highly preferred forward reactions in plants due to their mutations in either the left (lox44+lox75) or right (lox43+lox76) arms. In some forms, spacers or arms are modified to remove start codons.

By combining these validated arm mutations with the specific spacers, new recombinase site pairs were generated. These new configurations were designated as KitL+KitR, KatL+KatR, KitLm3+KitRm3, and KatLm7+KatRm7 to distinguish from their predecessors. These names reflect their unique combinations of spacer specificity and arm mutations, forming the foundation of a modular toolkit for iterative, predictable recombinase-mediated cassette exchange in planta.

TABLE 1
Sequences of loxP sites and their origins

| | Name | Left Arm | Spacer | Right Arm | SEQ ID No. |
|---|---|---|---|---|---|
| Wt loxP | wt loxP | ATAACTTCGTATA | GCATACAT | TATACGAAGTTAT | 2 |
| from Sauer | | | | | |
| & | | | | | |
| Henderson, | | | | | |
| 1989: | | | | | |
| Spacers | double mutant | cctAgTTCcTATA | GCCTACAT | TATAgGAtGgagg | 3 |
| from Zhu | 1LR | | | | |
| et al., | double mutant | AgtAgaaCGTATA | GgcTAatT | TATACGAtGTagg | 4 |
| 2017a: | 2LR | | | | |
| Spacers | m3 | ATAACTTCGTATA | tggTAtta | TATACGAAGTTAT | 5 |
| from | m7 | ATAACTTCGTATA | ttcTAtcT | TATACGAAGTTAT | 6 |
| Langer, et | | | | | |
| al., 2002: | | | | | |
| Arms from | lox75 | taccgggCGTATA | GCATACAT | TATACGAAGTTAT | 7 |
| Albert et | lox76 | ATAACTTCGTATA | GCATACAT | TATACGcccggta | 8 |
| al., 1995a: | lox44 | AatgCaTgcTATA | GCATACAT | TATACGAAGTTAT | 9 |
| | lox43 | ATAACTTCGTATA | GCATACAT | TATAgGTAccgAg | 10 |
| BioPop | KitL | taccgggCGTATA | GCcTACAT | TATACGAAGTTAT | 11 |
| Landing | KitR | ATAACTTCGTATA | GCcTACAT | TATACGcccggta | 12 |
| Pad: | KatL | AataCaTCGTATA | GgcTAatT | TATACGAAGTTAT | 13 |
| | KatR | ATAACTTCGTATA | GgcTAatT | TATAgGTAccgAg | 14 |
| | KitLm3 | taccgggCGTATA | tggTAtta | TATACGAAGTTAT | 43 |
| | KitRm3 | ATAACTTCGTATA | tggTAtta | TATACGcccggta | 44 |
| | KatLm7 | AataCaTCGTATA | ttcTAtcT | TATACGAAGTTAT | 45 |
| | KatRm7 | ATAACTTCGTATA | ttcTAtcT | TATAgGTAccgAg | 46 |

### Transient Assay

In transient leaf infiltration assays in N. benthamiana, excision frequency is visually assessed three days post-transformation, allowing time for Cre recombination and marker expression. A 2×CsVMV promoter (Li et al., 2001) resides upstream of the wtloxP to drive the expression of YFP (Miyawaki et al., 1997) initially. Constitutively driven Cre should excise the wtloxP-wtloxP region, AmCyan (Guryev et al., 2012), to allow for AmCyan expression driven by the same 2×CsVMV promoter. This assay helps test compatibility (ability of each pair to recombine as expected) and specificity (inability to recombine outside of the pairs' designated partner) of the spacers. In an exemplary assay, YFP expression is observed following initial transformation and selection, then constitutive Cre excises the wtloxP-wtloxP region, leaving a single recombined wtloxP. An exemplary loxP excision flowpath for transient assays is set forth in FIG. 6A.

### Stable Assay

In stable infiltration assays a heat shock promoter from Glycine max, Gmhsp17.6L (B. Wang et al., 2018), is utilized in the recombinase mediated cassette exchange to incorporate donor constructs. This promoter has previously driven successful recombinase expression in INRA 353-53 (P. tremula×P. tremuloides). The promoter was cloned into constructs to verify its function in planta. In stable transformations, initial insertion of the construct is validated by an antibiotic resistance cassette (StubiP: nptII: StubiT) (Garbarino & Belknap, 1994; Miroshnichenko et al., 2020; Yenofsky et al., 1990) was positioned at the end of the construct. Since stable transformations should have more controlled regulation of Cre, the heat-shock promoter, GmHsP17.6L is used to drive Cre expression. Therefore, when transformants are exposed to 42° C., Cre is expressed. This leads to the excision of the loxP-loxP region, which allows for AmCyan (Guryev et al., 2012) to be visible. If YFP fluorescence stops and AmCyan is not seen after heat induction, it could indicate methylation silencing rather than excision, which has been observed in poplar (Liu et al., 2021). Using a second visual marker, AmCyan, will help identify excision events and confirm excision frequency compared to silencing (no fluorescence) or improper Cre induction/loxP recognition (YFP fluorescence seen after heat treatment). In an exemplary assay, antibiotic and visual selections nptII and YFP respectively are induced following the first transformation, then induction of Cre with heat excises the loxP-loxP region leaving a recombined loxP in its place and the 2×CsVMV promoter that was driving YFP will instead drive AmCyan.

Made in Biorender, an exemplary loxP excision flowpath for transient assays is set forth in FIG. 6B.

### Compatibility and Specificity

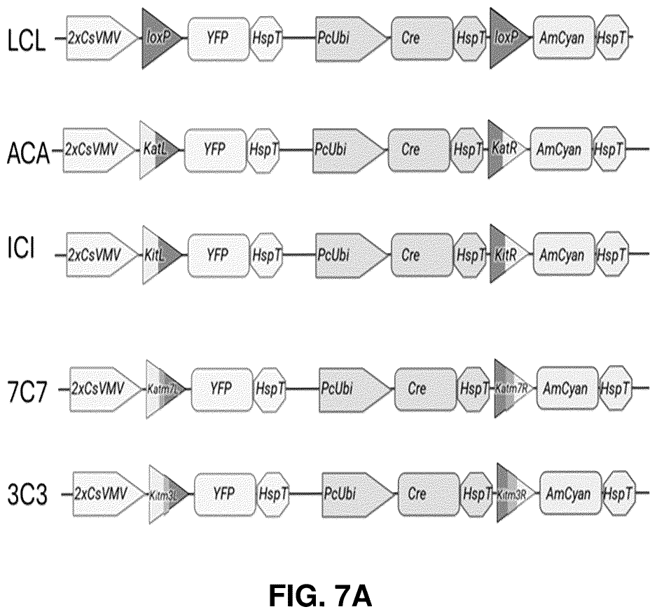

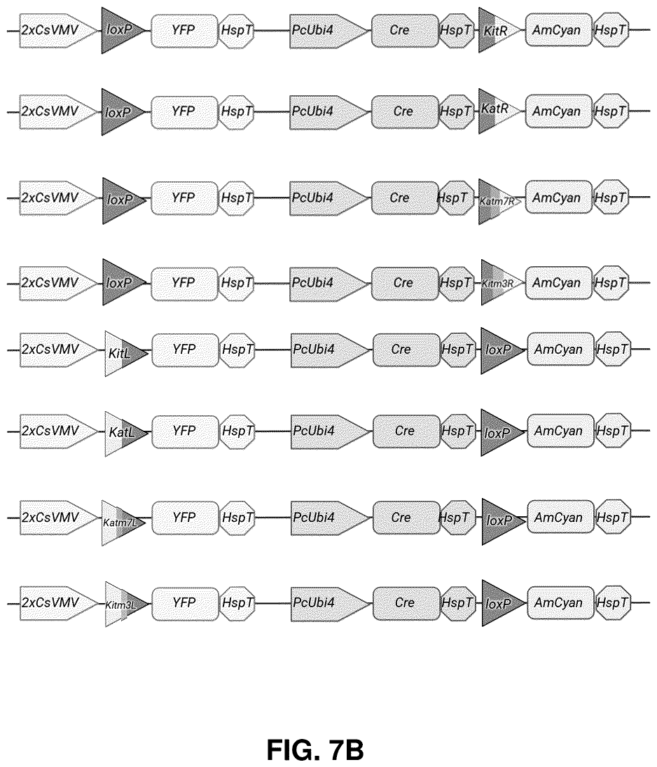

After initial testing done with wild-type loxP, the KitL+KitR, KatL+KatR, KitLm3+KitRm3, KatLm7+KatRm7 mutant combinations were tested to confirm their compatibility/ability to recombine as expected in N. benthamiana and to test their specificity/inability to recombine outside of their pair. A panel including all possible compatibility tests based on the 4 new pairs of recombinase sites is depicted in FIG. 7A.

To test specificity between the mutant loxP pairs outside of their respective partners a panel including all possible compatibility tests based on the 4 new pairs of recombinase sites is depicted in FIG. 7B. To eliminate unnecessary work, these specificity tests were conducted before testing the specificity between the new recombinase sites in case they are not specific enough. None of these are expected to allow for excision and therefore YFP is expected to continue to fluoresce after heat treatment.

### Visual, Antibiotic and PCR Confirmation

For transient assays, leaf discs from infiltrated tissue are imaged, and YFP and AmCyan fluorescence are quantified using a Biotek Synergy H1 Reader.

In stable transformations, transformants are selected on the appropriate antibiotics. Fluorescence imaging at the callus stage, together with line PCR, is then used to confirm proper recombination. Line PCR is a rapid technique that uses fishing line to collect DNA for PCR in place of traditional extraction. It has been validated in Arabidopsis thaliana (Lynagh et al., 2023) and successfully implemented in poplar in our lab (data not published). This method can be applied to confirm excision and insertion events directly at the callus stage, providing results significantly earlier than waiting for regenerated plants in poplar transformations.

### Development of Iterative Recombination-Based Cassette Exchange (RMCE) in Poplar Using the Established Toolkit

Upon validation of the sites and the heat shock promoter construct in poplar, the construction of a landing pad for iterative insertion was transformed into poplar 717. The initial insertion contained a wt loxP and a KitR, serving as the primary target for recombination. The left arm of the wt loxP and the right arm of the KitR should remain stationary, recombining with wt loxP and KatR, respectively. This configuration permits the insertion of a cassette containing a third recombination site (RS) that is incompatible with the existing recombinase pairs. Given that each pair of sites is expected to only recombine with its specific partner due to the specificity of the spacers, the insertion process is unidirectional, precluding alternative arrangements.

An iterative, four step RMCE was proposed, as follows:
Step i: The initial transformation will contain YFP and nptII selection as well as the landing pad composed of loxP and KitR. The black line that the construct is on represents the genome;
Step ii: The first donor for RMCE begins with wt loxP and ends with KitL. It contains the visual marker Amcyan that lacks a promoter. There is room for a gene of interest (GOI) to be between the terminator of AmCyan and the KitL;
Step iii: When KitR and KitL recombine, they will form KitLR which is a double mutant “loxbox” After the first RMCE, the 2×CsVMV which was driving YFP would now drive AmCyan;

Step iv: The second donor will contain a YFP that lacks a promoter. When the donor is inserted with the second gene of interest and YFP, the upstream 2×CsVMV will drive the YFP. Alternating between the AmCyan and YFP markers allows for visual aid in selecting the RMCE events; and
Step v: The resulting recombination leaves two “Loxboxes” in the genome and the same landing pad as the first insertion, thus allowing the cycle to begin again.

Elements between KatR and KitL in the donor cassette will be preserved and integrated into the genome. The recombination of KitL with KitR will result in a KitL/R “Loxbox” sequence remaining in the genome.

Elements positioned between KatR and KitL in the donor cassette are preserved and integrated into the genome. Recombination of KitL with KitR yields a KitL/R “Loxbox” sequence that remains in the genome. As illustrated in FIGS. 8A-8C, traits of interest can be inserted between the AmCyan terminator and KitL on the donor plasmid, ensuring their integration alongside AmCyan. Subsequent insertions alternate between YFP and AmCyan markers. Previous marker sequences remain in the genome but lack promoters and therefore should not be expressed.

Insertion of KatR into the target functions analogously to KitR in the preceding reaction, enabling further rounds of integration. This “Trojan horse” strategy-introducing a new RS site with each insertion-supports multiple iterative events, overcoming the limitations of earlier methods (Nandy et al., 2015; Pathak et al., 2022).

### Evaluation of Iterative RMCE

Alternating visual markers (YFP and AmCyan) provide a direct means of selecting successful RMCE events. In addition to visual screening, an alternative donor cassette containing HPH (hygromycin resistance gene) in place of AmCyan has been cloned to enable antibiotic selection. Insertions are confirmed by PCR, which also addresses concerns that upstream terminators may not fully block expression of previous markers.

Recombination frequency is determined either by PCR in stable transformants or by visual confirmation of marker switching in transient assays. These data allow estimation of the number of screens required to identify recombinants in systems lacking visual markers.

### Results

Preliminary promoter comparison assays in tobacco established the 2×CsVMV promoter (Li et al., 2001) as a strong and reliable driver of expression. This promoter was further validated in poplar by fusing GFP to nptII with a histag bridge, creating a bifunctional marker. The resulting calli grew robustly on kanamycin (50 mg/mL) and have been maintained for over a year. Inspired by this success, attempts were made to fuse visual and antibiotic markers to simplify the landing pad. However, AmCyan fused to HPH displayed reduced fluorescence compared to AmCyan alone, and YFP fused to nptII was non-functional, leading to discontinuation of this approach.

To confirm that the interruption of the loxPs between the promoter and the coding sequence would not interfere with expression of the fluorescent protein, interrupting the expression with wt loxP, KitL and KatL was tested. All cases led to YFP expression in tobacco. Each construct was repeated with three plants and three leaves infiltrated per plant.

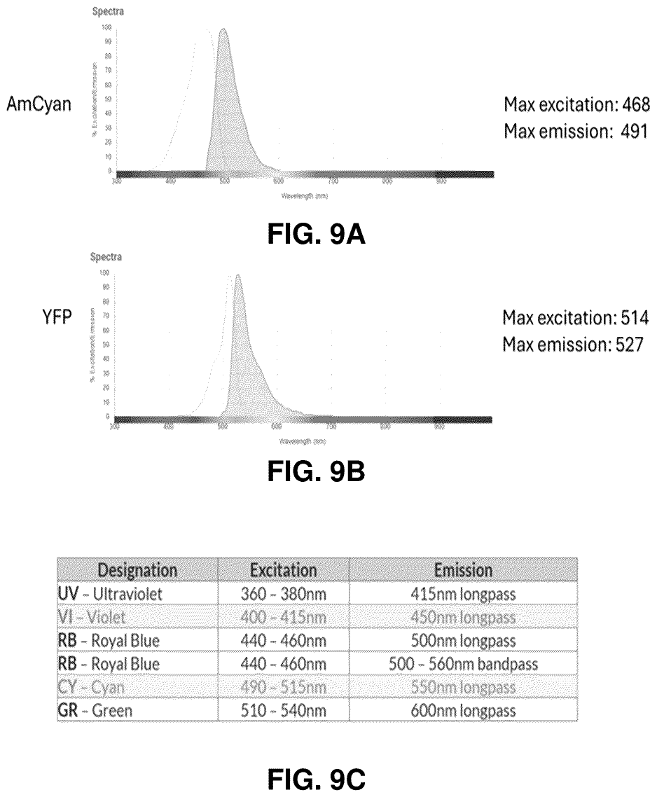

Distinct fluorescence of YFP and AmCyan was confirmed in poplar using NIGHTSEA wavelength sets, with excitation/emission spectra demonstrating clear separation (FIGS. 9A-9C).

Heat-shock assays validated the soybean-derived GmHsP17.6L promoter as an effective inducible system in poplar. AmCyan expression was induced after 42° C. treatment, confirming its suitability for Cre regulation in applications requiring inducible expression; the plate before heatshock showed the control AmCyan expression but, a GmHsP17.6L-driven AmCyan was not expressed prior to heatshock. 1 hour after the 24 hour 42° C. heatshock, the GmHsP17.6L-driven AmCyan expression is comparable to the control.

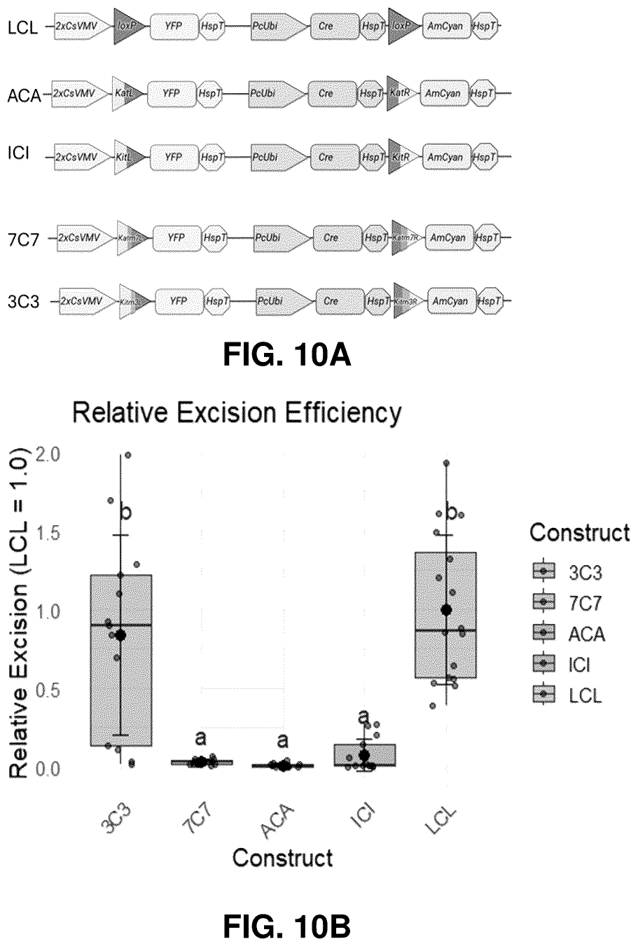

Toolkit evaluation through transient assays in N. benthamiana revealed important insights. Plants were transfected (OD600=0.600-0.800) with five constructs testing for compatibility (FIG. 17). After 3 days, the leaves were imaged and hole punches were taken for quantifying YFP and AmCyan with a Biotek Synergy H1 Reader from Agilent. The YFP and AmCyan values were standardized by subtracting the baseline fluorescence of untransformed leaves infiltrated with buffer alone. Then the excision values were calculated by diving the AmCyan values by the YFP values. This was then normalized to the wt loxP construct (LCL) since it is expected to have the highest excision rate without any mutations present in the recombinase sites. The compatibility testing showed that KitLm3+KitRm3 performed comparably to wt loxP, while KatLm7+KatRm7 and KatL+KatR exhibited very low compatibility. KitL+KitR were moderately compatible, less than KitLm3/Rm3 but more than the Kat pairs (FIGS. 10A-10B). A second trial is scheduled for Fall 2025, with stable transformations planned for Winter 2025.

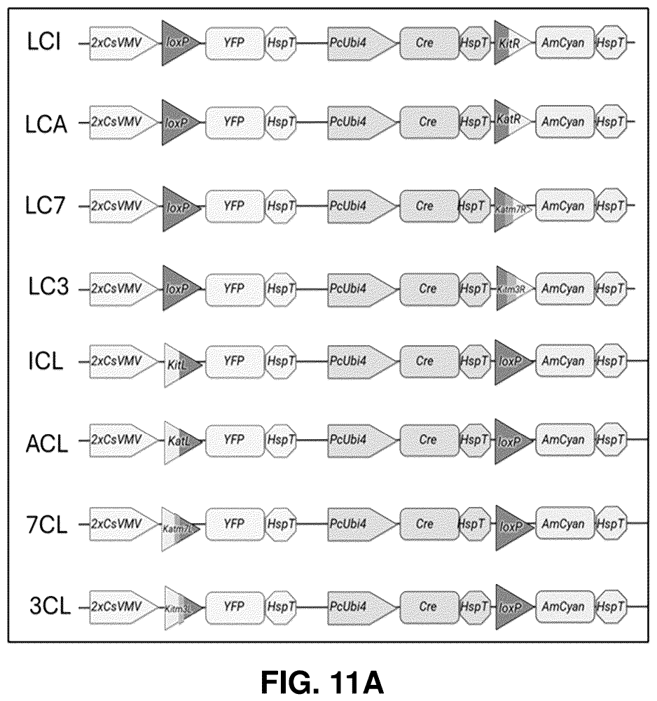

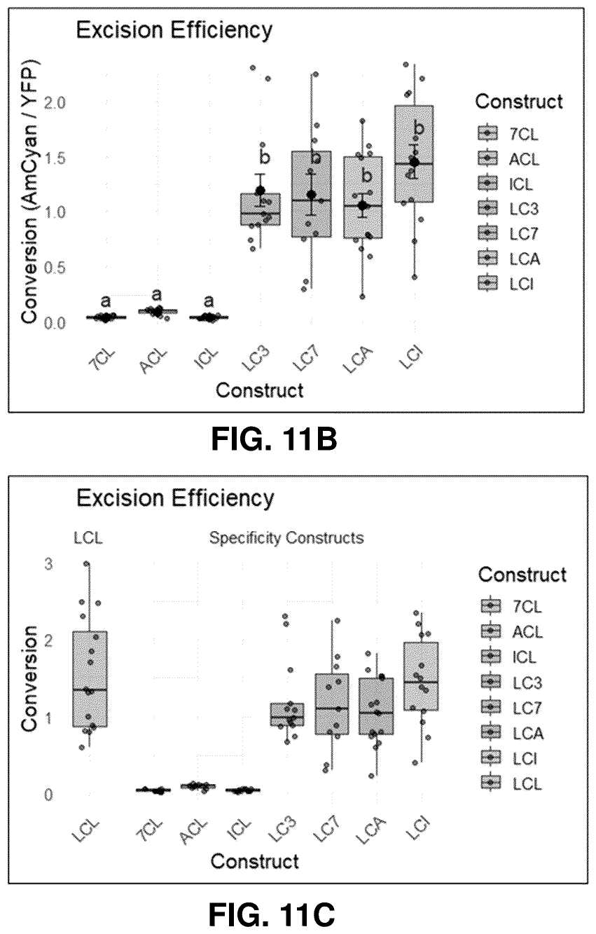

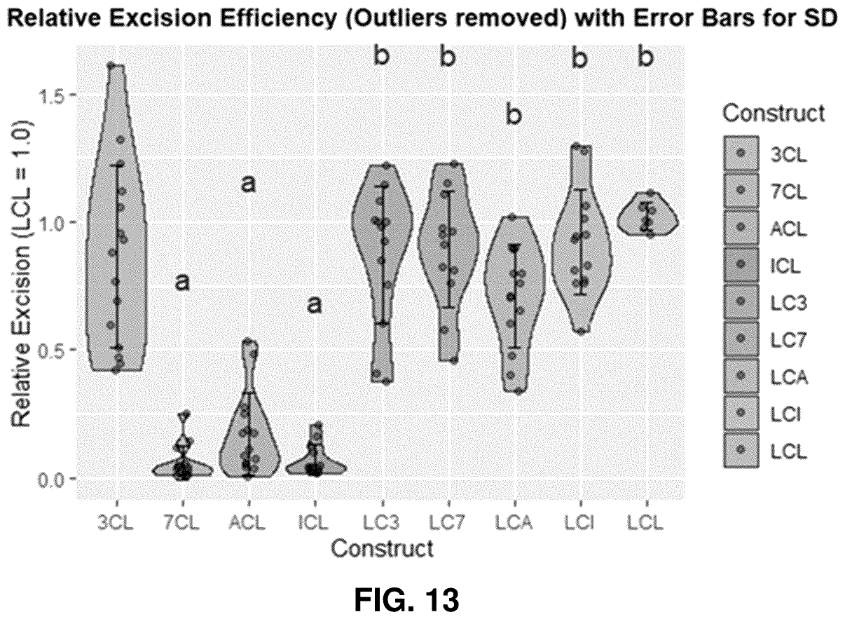

Specificity assays highlighted a potential limitation: mutant loxP pairs recombined with wt loxP under certain configurations, producing AmCyan expression when wt loxP preceded the mutant site (FIGS. 11A-11C, 13). Future trials will include additional controls (LCL) to refine these observations. Since there was no LCL control, the samples were standardized to the baseline untransformed plants, but their excision frequencies were not normalized. However, even when including the LCL data from the compatibility tests (which is not a true control since they were transformed on different days, but should provide some perspective), the excision frequency indicates that the pair are not as specific as desired when allowed to recombine with wt loxP.

If the new spacers were specific within the pairs, there would be no excision and therefore no AmCyan expression. However, this was not the case that was observed. Even though the publications describing the m3 and m7 spacer specifically said recombination between them and the wt loxP was not possible. This suggests that spacer specificity may depend on positional context in planta.

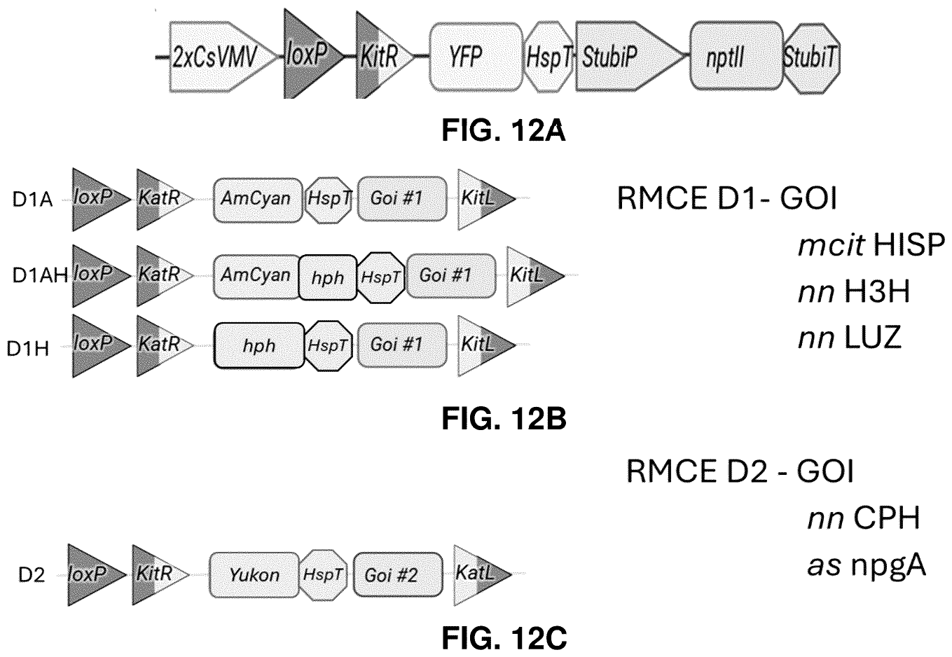

RMCE testing is ongoing. Poplar 717 transformed with the landing pad construct (FIG. 12A) has been propagated for donor cassette exchange experiments. Plantlets derived from this transformation in March 2025 have been confirmed by visual and antibiotic selection.

Donor constructs currently under evaluation include AmCyan alone, AmCyan fused to HPH, and HPH alone (FIGS. 12B-12C). Each carries genes of interest conferring dim luminescence, which will be amplified by subsequent donor insertions (Khakhar et al., 2020). A second donor cassette, originally intended to include YFP, was redesigned to incorporate a different fluorescent protein to facilitate PCR confirmation. This transformation was initiated in November 2025, with data forthcoming. Parallel transient assays in N. benthamiana are planned to corroborate results and are scheduled for transformation in Winter 2025.

### Example 2: Development of LOXBOX Cassette System with Increased Accessibility

T-DNA (transfer DNA) is a segment of the Ti (tumor-inducing) or Ri (root-inducing) plasmid in Agrobacterium species, specifically defined by 25-bp right and left border repeats. It is transferred to plant cells and integrated into their genome, serving as a vital tool for genetic engineering and insertion mutagenesis.

Iterative plant transformations that rely on Agrobacterium-mediated delivery require T-DNA borders to insert donor constructs into the genome. Once a donor is integrated, it can in principle recombine with a pre-installed landing pad via RMCE. However, the donor's physical accessibility becomes a major bottleneck: because T-DNA integrates at random genomic positions, the proximity to the landing pad is expected to be suboptimal for recombination. This positional dependence limits the reliability of iterative gene stacking workflows that depend on donor: landing pad interactions.

### Methods

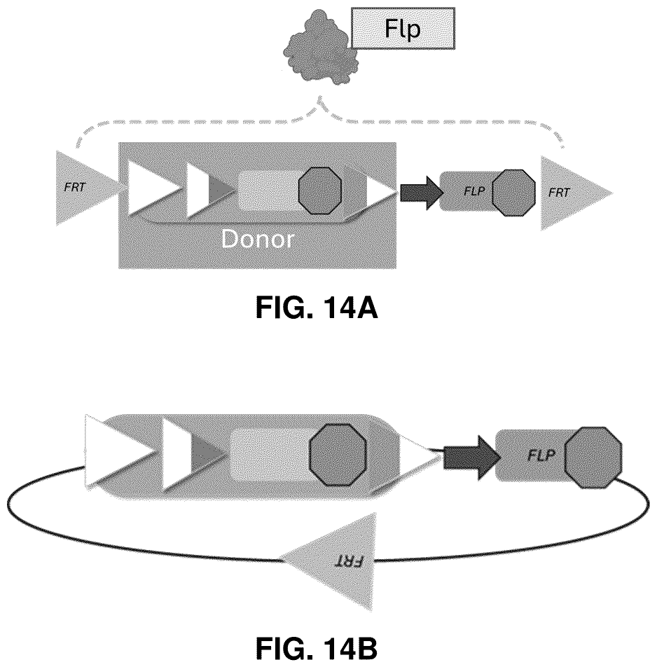

One proposed solution to increase donor accessibility using Flp/FRT excision by flanking the donor cassette with FRT sites, the donor can be excised from its chromosomal context upon FLP expression, which would then allow the donor to exist as an extrachromosomal circle, thus improving accessibility. The Flp/FRT system is a site-specific recombination tool derived from yeast, used to excise, invert, or swap DNA sequences flanked by 34-bp FRT (FLP recognition target) sites using the Flp recombinase. The Flp enzyme binds to FRT sites and catalyzes a precise cut-and-paste, removing the intervening sequence and leaving behind a single scarred FRT site. The proposed donor and FLP constructs flanked by FRT sites then self-excise after FLP expression, to create an extrachromosomal plasmid-like DNA (see FIGS. 14A-14B).

Another proposed solution to increase donor accessibility includes and enrichment uses viral replicons. Viral replicons offer a different way to deliver donor DNA that uses geminiviral replicon sites to leverage rolling-circle replication to generate high-copy, extrachromosomal plasmid-like circular DNA fragments. Geminiviral replicons have been used in plant transformation to increase guides for CRISPR editing, but not for donor enrichment in a RMCE platform.

### REFERENCES

Ainley, et al. (2013). Trait stacking via targeted genome editing. Plant Biotechnology Journal, 11(9). doi.org/10.1111/pbi.12107
Albert, et al. (1995). Site-specific integration of DNA into wild-type and mutant lox sites placed in the plant genome. The Plant Journal, 7 (4). doi.org/10.1046/j. 1365-313x.1995.7040649.x Bonawitz, et al. (2019). Zinc finger nuclease-mediated targeting of multiple transgenes to an endogenous soybean genomic locus via non-homologous end joining. Plant Biotechnology Journal, 17 (4). doi.org/10.1111/pbi.13012
Cody, et al. (2020). Site-specific recombinase genome engineering toolkit in maize. Plant Direct, 4 (3). doi.org/10.1002/pld3.209
Collier, et al. (2018). A versatile and robust Agrobacterium-based gene stacking system generates high-quality transgenic Arabidopsis plants. Plant Journal, 95 (4). doi.org/10.1111/tpj.13992
Dong, O. X., & Ronald, P. C. (2021). Targeted DNA insertion in plants. Proceedings of the National Academy of Sciences of the United States of America, 118 (22). doi.org/10.1073/pnas.2004834117
Garbarino, J. E., & Belknap, W. R. (1994). Isolation of a ubiquitin-ribosomal protein gene (ubi3) from potato and expression of its promoter in transgenic plants. Plant Molecular Biology, 24 (1). doi.org/10.1007/BF00040579
Hoersten, et al. (2024). Engineering spacer specificity of the Cre/loxP system. Nucleic Acids Research, 52 (13), 8017-8031. doi.org/10.1093/nar/gkae481
Kumar, et al. (2015). A modular gene targeting system for sequential transgene stacking in plants. Journal of Biotechnology, 207. doi.org/10.1016/j.jbiotec.2015.04.006
Khakhar, et al. (2020). Building customizable auto-luminescent luciferase-based reporters in plants. eLife, 9. doi.org/10.7554/elife.52786
Langer, et al. (2002). A genetic screen identifies novel non-compatible loxP sites. Nucleic Acids Research, 30 (14). doi.org/10.1093/nar/gkf421
Li, Z., Jayasankar, S., & Gray, D. J. (2001). Expression of a bifunctional green fluorescent protein (GFP) fusion marker under the control of three constitutive promoters and enhanced derivatives in transgenic grape (Vitis vinifera). Plant Science, 160 (5). doi.org/10.1016/S0168-9452 (01) 00336-3
Liu, et al. (2021). DNA methylation occurring in Cre-expressing cells inhibits loxP recombination and silences loxP-sandwiched genes. New Phytologist, 231 (1). doi.org/10.1111/nph.17353
Lynagh, et al. (2023). Letter to the Editor: Accurate Direct PCR with Arabidopsis and Rice. In Plant and Cell Physiology (Vol. 64).
McCue, et al. (2019). Transgene stacking in potato using the GAANTRY system. BMC Research Notes, 12 (1). doi.org/10.1186/s13104-019-4493-8
Miroshnichenko, et al. (2020). Evaluation of plant-derived promoters for constitutive and tissue-specific gene expression in potato. Plants, 9 (11). doi.org/10.3390/plants9111520
Missirlis, et al. (2006). A high-throughput screen identifying sequence and promiscuity characteristics of the loxP spacer region in Cre-mediated recombination. BMC Genomics, 7. doi.org/10.1186/1471-2164-7-73
Miyawaki, et al. (1997). Fluorescent indicators for Ca2+ based on green fluorescent proteins and calmodulin. Nature, 388 (6645). doi.org/10.1038/42264
Nandy, et al. (2015). Gene stacking in plant cell using recombinases for gene integration and nucleases for marker gene deletion. BMC Biotechnology, 15 (1). doi.org/10.1186/s12896-015-0212-2
Pathak, B., Nandy, S., & Srivastava, V. (2022). Multigene Transformation Through Cre-lox Mediated Site-Specific Integration in Rice. In Methods in Molecular Biology (Vol. 2408). doi.org/10.1007/978-1-0716-1875-2_19
Sauer, B., & Henderson, N. (1989). Cre-stimulated recombination at loxP-containing DNA sequences placed into the mammalian genome. Nucleic Acids Research, 17 (1). doi.org/10.1093/nar/17.1.147
Vergunst, et al. (1998). Site-specific integration of Agrobacterium T-DNA in Arabidopsis thaliana mediated by Cre recombinase. Nucleic Acids Research, 26 (11). doi.org/10.1093/nar/26.11.2729
Wang, et al. (2018). Heat-shock-induced removal of transgenes using the gene-deletor system in hybrid aspen (Populus tremula×P. tremuloides). Genes, 9 (10). doi.org/10.3390/genes9100484 Yarmolinsky, M., & Hoess, R. (2015). The Legacy of Nat Sternberg: The Genesis of Cre-lox Technology. In Annual Review of Virology (Vol. 2).
Yenofsky, et al. (1990). A mutant neomycin phosphotransferase II gene reduces the resistance of transformants to antibiotic selection pressure. Proceedings of the National Academy of Sciences of the United States of America, 87 (9). doi.org/10.1073/pnas.87.9.3435
Zhang, Z., & Lutz, B. (2002). Cre recombinase-mediated inversion using lox66 and lox71: method to introduce conditional point mutations into the CREB-binding protein. Nucleic acids research, 30 (17). doi.org/10.1093/nar/gnf089
Zhu, et al. (2017). Development of “Purple Endosperm Rice” by Engineering Anthocyanin Biosynthesis in the Endosperm with a High-Efficiency Transgene Stacking System. Molecular Plant, 10 (7). doi.org/10.1016/j.molp.2017.05.008

Unless defined otherwise, all technical and scientific terms used herein have the same meanings as commonly understood by one of skill in the art to which the disclosed invention belongs. Publications cited herein and the materials for which they are cited are specifically incorporated by reference. Those skilled in the art will recognize, or be able to ascertain using no more than routine experimentation, many equivalents to the specific embodiments of the invention described herein. Such equivalents are intended to be encompassed by the following claims.

We claim:

1. A modified plant genome comprising a mutant loxP landing pad nucleotide sequence, wherein the mutant loxP landing pad comprises
(i) a wild-type loxP nucleotide sequence,
optionally wherein the wild-type loxP comprises SEQ ID NO:1 or SEQ ID NO:2; and
(ii) a first mutant loxP nucleotide sequence;
optionally wherein the first mutant loxP nucleotide sequence comprises a nucleic acid sequence set forth in any one of SEQ ID NOs: 3-14 or 43-46.

2. The modified plant genome of claim 1, wherein the wild-type loxP nucleotide sequence is contiguous with the first mutant loxP nucleotide sequence.

3. The modified plant genome of claim 2, wherein the wild-type loxP nucleotide sequence comprises any one of SEQ ID NOs: 15-18.

4. A recombination mediated exchange cassette (RMEC) for genetic modification of the plant genome of claim 1, comprising
(i) a wild-type loxP nucleotide sequence,
optionally wherein the wild-type loxP comprises SEQ ID NO:1 or SEQ ID NO:2;
(ii) a second mutant loxP nucleotide sequence;
(iii) a donor nucleotide sequence; and
(iv) a third mutant loxP nucleotide sequence;
wherein the second and third mutant loxP nucleotide sequence(s) are orientated either side of the donor nucleotide sequence; and
wherein the wild-type loxP nucleotide sequence and the second mutant loxP nucleotide sequence are orientated upstream of the donor nucleotide sequence; and
wherein the third mutant loxP sequence is configured to direct Cre recombinase-mediated recombination with the first mutant loxP sequence within the landing pad of the modified plant genome.

5. The RMEC of claim 4, wherein each of the first, second and third mutant loxP sequences comprises a left arm region, a right arm region and a spacer sequence,
wherein each of the first, second and third mutant loxP sequences comprise a variation of a wildtype loxP sequence in a left arm region or in a right arm region,
optionally wherein each variation comprises a substitution of one or more nucleotides in the left arm region or the right arm region.

6. The RMEC of claim 4, wherein the donor nucleotide sequence comprises
(i) one or more gene,
optionally wherein the gene is exogenous to the target plant genome; and/or
(ii) a reporter gene.

7. The RMEC of claim 4, wherein the first mutant loxP nucleotide sequence comprises any one of SEQ ID NOs: 3, 5, 7, 9, 11, 13, 43, or 45; and
wherein the third mutant loxP nucleotide sequence comprises any one of SEQ ID NOs: 4, 6, 8, 10, 12, 14, 44, or 46; and
wherein the second mutant loxP nucleotide sequence is not the same as the first or third mutant loxP nucleotide sequence,
optionally wherein the second mutant loxP nucleotide sequence comprises any one of SEQ ID NOs: 3-14 or 43-46.

8. The RMEC of claim 7, wherein the first mutant loxP nucleotide sequence comprises SEQ ID NOs: 11 or 43; and
wherein the third mutant loxP nucleotide sequence comprises SEQ ID NOs: 12 or 44; and
optionally wherein the second mutant loxP nucleotide sequence comprises any one of SEQ ID NOs: 13, 14, 45, or 46.

9. The RMEC of claim 7, wherein
(i) the first mutant loxP nucleotide sequence comprises SEQ ID NO: 12; and
wherein the third mutant loxP nucleotide sequence comprises SEQ ID NO:11; and
optionally wherein the second mutant loxP nucleotide sequence comprises SEQ ID NO: 13; or
(ii) the first mutant loxP nucleotide sequence comprises SEQ ID NO:44; and
wherein the third mutant loxP nucleotide sequence comprises SEQ ID NO:43; and
optionally wherein the second mutant loxP nucleotide sequence comprises SEQ ID NO: 45.

10. The RMEC of claim 4, further comprising a promoter and/or a marker gene.

11. The RMEC of claim 10, comprising an AmCyan gene, or a YFP gene, or both, optionally wherein the AmCyan gene and/or the YFP gene is not operably associated with a promoter within the RMEC, but becomes operably linked to a promoter following recombination into a host plant genome.

12. A plant cell, plant tissue, plant material, plant part, or plant comprising the RMEC of claim 4.

13. A method for iterative stacking of donor genes within a target plant genome comprising
(a) contacting a target plant genome comprising a first mutant loxP landing pad with a first recombination mediated exchange cassette (RMEC), comprising
(i) a wild-type loxP nucleotide sequence,
optionally wherein the wild-type loxP comprises SEQ ID NO:1 or SEQ ID NO:2;
(ii) a second mutant loxP nucleotide sequence;
(iii) a donor nucleotide sequence; and
(iv) a third mutant loxP nucleotide sequence;
wherein the second and third mutant loxP nucleotide sequence(s) are orientated either side of the donor nucleotide sequence within the RMEC; and
wherein the wild-type loxP nucleotide sequence and the second mutant loxP nucleotide sequence are orientated upstream of the donor nucleotide sequence within the RMEC; and
wherein the third mutant loxP sequence is configured to direct Cre recombinase-mediated recombination with the first mutant loxP sequence within the first landing pad,
wherein the contacting occurs under conditions that enable Cre-recombinase mediated exchange of the RMEC into the first landing pad to provide a second mutant loxP landing pad comprising the second mutant loxP sequence; and
(b) optionally confirming recombination of the donor nucleotide sequence into the target genome.

14. The method of claim 13, further comprising
(c) contacting the target plant genome comprising the second mutant loxP landing pad with a second recombination mediated exchange cassette (RMEC), comprising
(i) a wild-type loxP nucleotide sequence,
optionally wherein the wild-type loxP comprises SEQ ID NO:1 or SEQ ID NO:2;
(ii) a fourth mutant loxP nucleotide sequence;
(iii) a donor nucleotide sequence; and
(iv) a fifth mutant loxP nucleotide sequence;
wherein the fourth and fifth mutant loxP nucleotide sequence(s) are orientated either side of the donor nucleotide sequence within the RMEC; and
wherein the wild-type loxP nucleotide sequence and the fifth mutant loxP nucleotide sequence are orientated upstream of the donor nucleotide sequence within the RMEC; and
wherein the fourth mutant loxP sequence is configured to direct Cre recombinase-mediated recombination with the second mutant loxP sequence within the second landing pad;
wherein the contacting occurs under conditions that enable Cre-recombinase mediated exchange of the RMEC into the second landing pad to provide a third mutant loxP landing pad comprising the fifth mutant loxP sequence; and
(d) optionally confirming recombination of the donor nucleotide sequence into the target genome.

15. The method of claim 14, further comprising
(e) repeating one or more of steps (a)-(d) once or more than once to iteratively recombine donor gene(s) into the target plant genome.

16. The method of claim 13, further comprising, prior to step (a), inserting within a target plant genome a first mutant loxP landing pad nucleotide sequence,
wherein the mutant loxP landing pad comprises
(i) a wild-type loxP nucleotide sequence,
optionally wherein the wild-type loxP comprises SEQ ID NO: 1 or SEQ ID NO:2; and
(ii) a first mutant loxP nucleotide sequence;
optionally wherein the first mutant loxP nucleotide sequence comprises a nucleic acid sequence set forth in any one of SEQ ID NOs: 3-14 or 43-46.

17. A modified plant cell, plant tissue, plant material, plant part, or plant prepared according to the method of claim 13.

18. A modified plant genome comprising a nucleic acid sequence comprising one or more heterologous genes derived from Cre-mediated recombination and one or more LoxBox sequences.

19. The modified plant genome of claim 18, wherein the one or more LoxBox sequence comprises any one of SEQ ID NOs: 39 or 40.

20. A modified plant cell, plant tissue, plant material, plant part, or plant comprising the modified plant genome of claim 18.
